SEQUENCE LISTING (23 sequences)

SEQ ID NO: 1 - protein, 404 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - protein, 135 aa, synthetic construct
MEWSGVFIFLLSVTAGVHSQVQLQQSGAELVRPGTSLKVSCKASGYAFTNYLIEWVNQRPGQGLEWIGVINPGSGGTHYNEKFKGKATLTADKSSSTAYMQLSSLTSDDSAIYFCARSRAVRAMDYWGQGTSVTV

SEQ ID NO: 3 - protein, 128 aa, synthetic construct
MESQTQVFVYMLLWLSGVDGDIVMTQSQKFMSTSVGDRVSVTCKASQNVGTNVAWYQQKPGQSPKSLIYSASYRYSGVPDRFTGSGSGTDFTLTISNVQSEDLAEYFCQQYNSYFFTFGAGTKLELKR

SEQ ID NO: 4 - protein, 8 aa, synthetic construct
GYAFTNYL

SEQ ID NO: 5 - protein, 8 aa, synthetic construct
INPGSGGT

SEQ ID NO: 6 - protein, 11 aa, synthetic construct
ARSRAVRAMDY

SEQ ID NO: 7 - protein, 6 aa, synthetic construct
QNVGTN

SEQ ID NO: 8 - skipped

SEQ ID NO: 9 - protein, 9 aa, synthetic construct
QQYNSYFFT

SEQ ID NO: 10 - protein, 141 aa, synthetic construct
MGWSSIIFFLVATATGVHSQVQLQQSGAELVRPGASVKISCKVSDYTFTDYVMHCVKQSHGKSLEWIGVISTYYGYSDYNQKFKGKATMTVDKSSSTAYMDLGRLTSEDSAVYYCARGGVTTATGAMDYWGQGTSVTVSSA

SEQ ID NO: 11 - protein, 142 aa, synthetic construct
MKSHTQVFISILLWLYGADGNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRADAAPTVSIFPPSS

SEQ ID NO: 12 - protein, 8 aa, synthetic construct
DYTFTDYV

SEQ ID NO: 13 - protein, 8 aa, synthetic construct
ISTYYGYS

SEQ ID NO: 14 - protein, 14 aa, synthetic construct
ARGGVTTATGAMDY

SEQ ID NO: 15 - protein, 6 aa, synthetic construct
ENVVTY

SEQ ID NO: 16 - skipped

SEQ ID NO: 17 - protein, 9 aa, synthetic construct
GQGYSYPYT

SEQ ID NO: 18 - protein, 78 aa, Homo sapiens
LVLKCKGAPKKPPQRLEWKLNTGRTEAWKVLSPQGGGPWDSVARVLPNGSLFLPAVGIQDEGIFRCQAMNRNGKETKS

SEQ ID NO: 19 - protein, 96 aa, Homo sapiens
PEIVDSASELTAGVPNKVGTCVSEGSYPAGTLSWHLDGKPLVPNEKGVSVKEQTRRHPETGLFTLQSELMVTPARGGDPRPTFSCSFSPGLPRHRA

SEQ ID NO: 20 - protein, 57 aa, Homo sapiens
PGGTVTLTCEVPAQPSPQIHWMKDGVPLPLPPSPVLILPEIGPQDQGTYSCVATHSS

SEQ ID NO: 21 - protein, 23 aa, Homo sapiens
MAAGTAVGAWVLVLSLWGAVVGA

SEQ ID NO: 22 - protein, 28 aa, Homo sapiens
RRQRRGEERKAPENQEEEEERAELNQSE

SEQ ID NO: 23 - protein, 402 aa, Mus musculus
MPAGTAARAWVLVLALWGAVAGGQNITARIGEPLVLSCKGAPKKPPQQLEWKLNTGRTEAWKVLSPQGGPWDSVARILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHEDEGTYSCVATHPSHGPQESPPVSIRVTETGDEGPAEGSVGESGLGTLALALGILGGLGVVALLVGAILWRKRQPRREERKAPESQEDEEERAELNQSEEAEMPENGAGGP

(19) United States
(12) Patent Application Publication
(10) Pub. No.: US 2025/0011424 A1
(43) Pub. Date: Jan. 9, 2025

(54) ANTIBODIES TO RECEPTOR OF ADVANCED GLYCATION END PRODUCTS (RAGE) AND USES THEREOF

(71) Applicant: SalvaRx LLC, Tortola (VG)

(72) Inventors: Cornelia Bentley, San Diego, CA (US); Jeff Pitman, San Diego, CA (US); Evan Holmes, San Diego, CA (US); Miguel de los Rios, San Diego, CA (US)

(21) Appl. No.: 18/711,742

(22) PCT Filed: Nov. 18, 2022
(86) PCT No.: PCT/US2022/080159

§ 371 Date: May 20, 2024

Related U.S. Application Data
(60) Provisional application No. 63/280,999, filed on Nov. 18, 2021.

(51) Int. Cl. C07K 16/28; A61K 39/00; A61P 35/00; G01N 33/68

(52) U.S. Cl. CPC C07K 16/2803; A61P 35/00; G01N 33/6854; A61K 2039/505; C07K 2317/33; C07K 2317/565; C07K 2317/76

(57) ABSTRACT

Disclosed herein are antibodies to receptor for advanced glycation end products (RAGE) and methods of using the antibodies.

### CROSS REFERENCE TO RELATED APPLICATIONS

The present application claims the benefit of U.S. Provisional Patent Application 63/280,999 filed Nov. 18, 2021, the entire contents of which are incorporated by reference herein.

### FIELD

The present disclosure relates to anti-RAGE antibodies and fragments thereof, antibody formulations, dosing regimens, and methods of using the same. The anti-RAGE antibodies and fragments thereof may be used in treatment and diagnosis of certain cancers, inflammatory states, and/or other conditions associated with increased RAGE expression or aberrant function.

### BACKGROUND

The receptor for advanced glycation end products (RAGE or AGER, see SEQ ID NO:1 or 23), is a multi-ligand receptor expressed on several cell types including immune cells such as myeloid-derived suppressor cells (MDSCs), tumor associated macrophages (TAMs) and mesenchymal stromal cells. The expression of RAGE is increased in tumor immune cell infiltrates and RAGE is also over-expressed on several tumor cell types.

RAGE also acts like a master switch from an acute inflammatory response to a chronic inflammatory response. When activated by one of several known ligands, cells are primed for anti-apoptotic activities and tumor cell proliferation. Activated RAGE creates a feed forward loop in which it enhances expression of itself and several known activating ligands.

Known ligands for RAGE include ligands belonging to the damage-associated molecular pattern (DAMP) which include high mobility group box 1 (HMGB1), S100 calcium binding proteins, amyloid fibrils, and nucleic acid backbones. Other ligands include those in the extracellular-signal-regulated kinase (Erk, also known as mitogen-activated protein kinase or MAPK) pathway, and ligands involved with regulating cell migration.

The chronic inflammatory state ignited by RAGE activation creates an immune suppressive environment through several mechanisms. These mechanisms include expression and activation of pro-inflammatory molecules such as tumor necrosis factor alpha (TNFα), interleukin 6 (IL-6), and matrix metalloproteinases (MMPs). RAGE also acts directly to suppress the activity of anti-tumoral lymphocytes through contact. RAGE activity recruits and promotes tumor infiltration with immune suppressive cells such as MDSCs and TAMs.

The present disclosure describes novel antibodies that bind to RAGE and can be used in therapeutic and/or diagnostic methods.

### SUMMARY

The disclosure is, in part, based on antibodies to RAGE. The disclosure also provides antibodies as therapeutic and diagnostic agents for use in targeting pathological conditions associated with expression and/or activity of RAGE. And, the disclosure provides methods, compositions, kits, and articles of manufacture related to RAGE.

The present disclosure provides binding molecules, in particular antibodies, which bind specifically to RAGE; representative anti-RAGE antibodies of the disclosure may comprise at least one of the antibody variable region amino acid sequences shown in SEQ ID NOs: 2, 3, 10, and 11, or individual CDRs thereof or related CDR sequences, as specified in more detail below.

Specifically, the present disclosure provides antibodies that bind to RAGE, more specifically monoclonal antibodies that bind to the V and/or C1-domain of RAGE. The present disclosure also provides RAGE-binding fragments that are at least 90%, 91%, 92%, 93%, 94%, 95%, 96%, 97%, 98%, 99%, or 100% identical to any of SEQ ID NOs: 2, 3, 10, and 11.

Included in the present disclosure are anti-RAGE antibodies that bind specifically to RAGE and comprise a light chain variable region having an amino acid sequence that is at least 90%, 91%, 92%, 93%, 94%, 95%, 96%, 97%, 98%, 99%, or 100% identical to any of SEQ ID NOs: 3, and 11, or is a RAGE-binding fragment or a homologous variant thereof, of an antibody comprised in said sequences. Also included in the present disclosure are anti-RAGE antibodies that bind specifically to RAGE and comprise an amino acid sequence that is at least 90%, 91%, 92%, 93%, 94%, 95%, 96%, 97%, 98%, 99%, or 100% identical to a CDR (as identified herein) comprised in SEQ ID NOs: 3 or 11.

Also included are anti-RAGE antibodies and fragments thereof that bind specifically to RAGE and comprise a heavy chain variable region having an amino acid sequence that is at least 90%, 91%, 92%, 93%, 94%, 95%, 96%, 97%, 98%, 99%, or 100% identical to any of SEQ ID NOs: 2, and 10, or is a RAGE-binding fragment or a homologous variant thereof, of an antibody comprised in said sequences. Also included in the present disclosure are anti-RAGE antibodies that bind specifically to RAGE and comprise an amino acid sequence that is at least 90%, 91%, 92%, 93%, 94%, 95%, 96%, 97%, 98%, 99%, or 100% identical to a CDR (as identified herein) comprised in SEQ ID NOs: 2 or 10.

The anti-RAGE antibodies of the disclosure include an anti-RAGE antibody or fragment thereof or a RAGE-binding fragment or homologous variant thereof as described above, which is selected from the group consisting of a chimeric antibody, a CDR-grafted or humanized antibody, a single chain antibody, a fusion protein, and a human antibody.

In certain embodiments, an antibody or an antibody fragment that binds to RAGE (SEQ ID NO: 1) or a fragment thereof is provided, wherein the antibody or fragment thereof comprises a variable domain comprising a heavy chain (VH) comprising the amino acid sequence of SEQ ID NO: 2 or 10 and a light chain (VL) comprising the amino acid sequence of SEQ ID NO: 3 or 11.

In certain embodiments, an antibody or an antibody fragment that binds to RAGE or a fragment thereof is provided, wherein the antibody or fragment thereof comprises a variable domain comprising at least one, two, three, four, five or six complementarity determining region (CDR) sequences comprising: CDR-H1 comprising the amino acid sequence of SEQ ID NO: 4 or 12; CDR-H2 comprising the amino acid sequence of SEQ ID NO: 5 or 13; and CDR-H3 comprising the amino acid sequence of SEQ ID NO: 6 or 14. In certain embodiments, the antibody or fragment thereof further comprises CDR-L1 comprising the amino acid sequence of SEQ ID NO: 7 or 15: CDR-L2 comprising the amino acid sequence of SEQ ID NO: 8 or 16; and CDR-L3 comprising the amino acid sequence of SEQ ID NO: 9 or 17. In certain embodiments, the antibody or fragment thereof comprises a variable domain comprising at least one, two, three, four, five or six complementarity determining region (CDR) sequences comprising amino acid sequences that are at least 90%, 91%, 92%, 93%, 94%, 95%, 96%, 97%, 98%, 99%, or 100% identical to any of SEQ ID NOs 4-9 or 12-17.

In certain embodiments, an antibody or an antibody fragment that binds to RAGE or a fragment thereof is provided, wherein the antibody or fragment thereof comprises a variable domain comprising CDR sequences comprising: CDR-H1 comprising the amino acid sequence of SEQ ID NO: 4 or 12; CDR-H2 comprising the amino acid sequence of SEQ ID NO: 5 or 13; and CDR-H3 comprising the amino acid sequence of SEQ ID NO: 6 or 14. In certain embodiments, the antibody or fragment thereof further comprises CDR-L1 comprising the amino acid sequence of SEQ ID NO: 7 or 15: CDR-L2 comprising the amino acid sequence of SEQ ID NO: 8 or 16; and CDR-L3 comprising the amino acid sequence of SEQ ID NO: 9 or 17. In certain embodiments, the antibody or fragment thereof comprises a variable domain comprising CDR sequences comprising amino acid sequences that are at least 90%, 91%, 92%, 93%, 94%, 95%, 96%, 97%, 98%, 99%, or 100% identical to any of SEQ ID NOs 4-9 or 12-17.

In one embodiment, an antibody or an antibody fragment that binds to RAGE or a fragment thereof is provided, wherein the antibody comprises a VH comprising the amino acid sequence of SEQ ID NO: 2. In certain embodiments, the antibody or fragment thereof further comprises a VL comprising the amino acid sequence of SEQ ID NO: 3. In certain embodiments, the antibody or fragment thereof comprises a VH comprising the amino acid sequence of SEQ ID NO: 2 and a VL comprising the amino acid sequence of SEQ ID NO: 3. In certain embodiments, the antibody comprises a VH comprising an amino acid sequence that is at least 90%, 91%, 92%, 93%, 94%, 95%, 96%, 97%, 98%, 99%, or 100% identical to SEQ ID NO: 2 and/or a VL comprising an amino acid sequence that is at least 90%, 91%, 92%, 93%, 94%, 95%, 96%, 97%, 98%, 99%, or 100% identical to SEQ ID NO: 3. In certain embodiments, the antibody or fragment thereof comprises a variable domain comprising at least one CDR sequence comprising amino acid sequences that are at least 90%, 91%, 92%, 93%, 94%, 95%, 96%, 97%, 98%, 99%, or 100% identical to any of SEQ ID NOs 4-9.

In one embodiment, an antibody or an antibody fragment that binds to RAGE or a fragment thereof is provided, wherein the antibody or fragment thereof comprises a VH comprising the amino acid sequence of SEQ ID NO: 10. In certain embodiments, the antibody or fragment thereof further comprises a VL comprising the amino acid sequence of SEQ ID NO: 11. In certain embodiments, the antibody or fragment thereof comprises a VH comprising the amino acid sequence of SEQ ID NO: 10 and a VL comprising the amino acid sequence of SEQ ID NO: 11. In certain embodiments, the antibody comprises a VH comprising an amino acid sequence that is at least 90%, 91%, 92%, 93%, 94%, 95%, 96%, 97%, 98%, 99%, or 100% identical to SEQ ID NO: 10 and/or a VL comprising an amino acid sequence that is at least 90%, 91%, 92%, 93%, 94%, 95%, 96%, 97%, 98%, 99%, or 100% identical to SEQ ID NO: 11. In certain embodiments, the antibody or fragment thereof comprises a variable domain comprising at least one CDR sequence comprising amino acid sequences that are at least 90%, 91%, 92%, 93%, 94%, 95%, 96%, 97%, 98%, 99%, or 100% identical to any of SEQ ID NOs 12-17.

In one embodiment, an antibody or an antibody fragment that binds to RAGE or a fragment thereof is provided, wherein the antibody or fragment thereof comprises:
(1) a CDR-H1 comprising the amino acid sequence of SEQ ID NO: 4;
(2) a CDR-H2 comprising the amino acid sequence of SEQ ID NO: 5;
(3) a CDR-H3 comprising the amino acid sequence of SEQ ID NO: 6;
(4) a CDR-L1 comprising the amino acid sequence of SEQ ID NO: 7;
(5) a CDR-L2 comprising the amino acid sequence of SEQ ID NO: 8; and
(6) a CDR-L3 comprising the amino acid sequence of SEQ ID NO: 9.

In certain embodiments, the antibody or antibody fragment comprises amino acid sequences that are at least 90%, 91%, 92%, 93%, 94%, 95%, 96%, 97%, 98%, 99%, or 100% identical to any of SEQ ID NOs 4-9.

In one embodiment, an antibody or an antibody fragment that binds to RAGE or a fragment thereof is provided, wherein the antibody or fragment thereof comprises:
(1) a CDR-H1 comprising the amino acid sequence of SEQ ID NO: 12;
(2) a CDR-H2 comprising the amino acid sequence of SEQ ID NO: 13;
(3) a CDR-H3 comprising the amino acid sequence of SEQ ID NO: 14;
(4) a CDR-L1 comprising the amino acid sequence of SEQ ID NO: 15;
(5) a CDR-L2 comprising the amino acid sequence of SEQ ID NO: 16; and
(6) a CDR-L3 comprising the amino acid sequence of SEQ ID NO: 17.

In certain embodiments, the antibody or antibody fragment comprises amino acid sequences that are at least 90%, 91%, 92%, 93%, 94%, 95%, 96%, 97%, 98%, 99%, or 100% identical to any of SEQ ID NOs 12-17.

In certain embodiments, an antibody or an antibody fragment that binds to RAGE or a fragment thereof is provided, wherein the antibody or fragment thereof comprises (a) a VH sequence having at least 95% sequence identity to the amino acid sequence of SEQ ID NO: 2, or SEQ ID NO: 10; or (b) a VL sequence having at least 95% sequence identity to the amino acid sequence of SEQ ID NO: 3, or SEQ ID NO: 11.

In one embodiment, an antibody or an antibody fragment that binds to RAGE or a fragment thereof is provided, wherein the antibody or fragment thereof comprises a VH sequence of SEQ ID NO: 2 and a VL sequence of SEQ ID NO: 3. In another embodiment, an antibody or an antibody fragment that binds to RAGE or a fragment thereof is provided, wherein the antibody or fragment thereof comprises a VH sequence of SEQ ID NO: 10 and a VL sequence of SEQ ID NO: 11.

In certain embodiments, an antibody or an antibody fragment that binds to RAGE or a fragment thereof is provided, wherein the antibody binds to an epitope within a fragment of RAGE. In certain embodiments, an antibody or an antibody fragment that binds to RAGE or a fragment thereof is provided, wherein the antibody or fragment thereof binds to an epitope within a fragment of RAGE comprising the V and/or C1 domains of human RAGE amino acid sequence of SEQ ID NO: 1. The V domain of human RAGE comprises amino acids 23-116 of SEQ ID NO: 1 (also provided as SEQ ID NO: 18), while the C1 domain comprises amino acids 124-221 of SEQ ID NO: 1 (also provided as SEQ ID NO: 19) and the C2 domain comprises amino acids 227-317 of SEQ ID NO: 1 (also provided as SEQ ID NO: 20).

In certain embodiments, the anti-RAGE antibody is a monoclonal antibody. In certain embodiments, the anti-RAGE antibody is humanized. In certain embodiments, the anti-RAGE antibody is a human antibody. In certain embodiments, at least a portion of the framework sequence of the anti-RAGE antibody is a human consensus framework sequence or human germline sequence. In one embodiment, the antibody is an antibody fragment selected from a Fab, Fab′-SH, Fv, scFv, or (Fab′)₂ fragment.

In one aspect, a nucleic acid encoding any of the above anti-RAGE antibodies or fragments thereof is provided. In one embodiment, a vector comprising the nucleic acid is provided. In one embodiment, the vector is an expression vector. In one embodiment, a host cell comprising the vector is provided. In one embodiment, the host cell is eukaryotic. In another embodiment, the host cell is mammalian. In yet another embodiment, the host cell is prokaryotic. In one embodiment, a method of making an anti-RAGE antibody or fragment thereof is provided, wherein the method comprises culturing the host cell under conditions suitable for expression of the nucleic acid encoding the antibody, and isolating the antibody. In certain embodiment, the method further comprises recovering the anti-RAGE antibody or fragment thereof from the host cell. In certain embodiments, a composition comprising any of the anti-RAGE antibodies or fragments thereof described herein is provided. In one embodiment, the composition further comprises a pharmaceutically acceptable carrier.

In one aspect, provided herein is a pharmaceutical composition comprising an anti-RAGE antibody or fragment thereof. In certain embodiments, the composition is suitable for subcutaneous administration. In certain embodiments, the composition is suitable for intravenous administration. In certain embodiments, the viscosity of the composition is less than about 10 cP at 25° C. In other embodiments, the viscosity of the composition is less than about 20 cP at 25° C., such as for example and not limitation, about 15 cP, about 16 cP, about 17 cP, about 18 cP, and about 19 cP. Any anti-RAGE antibodies known in the art or described herein may be formulated into the composition.

In one aspect, provided herein is a subcutaneous administration device containing an anti-RAGE antibody or fragment thereof or a composition comprising an anti-RAGE antibody or fragment thereof described herein. In certain embodiments, the device is for delivering to an individual a flat dose in the range of about 200 to about 1200 mg of the antibody.

In one aspect, the disclosure concerns methods of inhibiting RAGE mediated activation of the Erk/MAPK or p53 pathways in a subject, said method comprising administering to the subject an effective amount of any of the anti-RAGE antibodies or fragments thereof described herein. In another aspect, the disclosure concerns methods of reducing at least one of chronic inflammation, immune suppression, cancer/tumor cell migration, cancer/tumor cell infiltration, suppressive immune cell infiltration into the tumor, tumor promoting immune cell infiltration, cancer/tumor growth, liver damage and fibrosis, neovascularization in the eye, respiratory diseases, infections, and/or cancer/tumor progression in a subject, said method comprising administering to the subject an effective amount of any of the anti-RAGE antibodies or fragments thereof described herein.

In one aspect, the disclosure concerns methods of inhibiting RAGE mediated activation of the Erk/MAPK or p53 pathways in a subject, said method comprising administering to the subject an effective amount of any of the anti-RAGE antibodies or fragments thereof described herein. In another aspect, the disclosure concerns methods of increasing at least one of CD8+ T cell infiltration, M1 TAM infiltration or polarization from M2 to M1 TAMs, NK T cells, NK cells, CD8+ anti-tumoral memory cells, increased M1 function (such as increased production of IL-27), said method comprising administering to the subject an effective amount of any of the anti-RAGE antibodies or fragments thereof described herein.

In one aspect, the disclosure concerns methods of inhibiting RAGE activity to prevent and/or treat cancer, such as for example and not limitation cancers characterized by high vascularization such as colorectal and renal cancers, said method comprising administering to the subject an effective amount of any of the anti-RAGE antibodies or fragments thereof described herein.

In another aspect, the disclosure concerns methods of inhibiting RAGE activity to alter a tumor microenvironment, such as for example and not limitation, to inhibit vascularization to a tumor, inhibit growth of the tumor, inhibit or decrease expression of growth factors and/or inflammatory factors within the tumor, decrease monocytic myeloid derived suppressor cells (MDSC), decrease inflammatory monocytes (iMonos), decrease M2 tumor associated macrophage (M2 TAM), increase M1 macrophage and M1 macrophage signaling, increase M1/M2 macrophage tumor infiltrates and/or signaling, M2 to M1 polarization, and/or to promote T cell activity and infiltration into the tumor, said method comprising administering to the subject an effective amount of any of the anti-RAGE antibodies or fragments thereof described herein.

In another aspect, the disclosure concerns methods of inhibiting RAGE activity to prevent the development of chronic inflammation, said method comprising administering to the subject an effective amount of any of the anti-RAGE antibodies or fragments thereof described herein. In a related aspect, the disclosure concerns methods of inhibiting RAGE activity to maintain an acute inflammatory state, said method comprising administering to the subject an effective amount of any of the anti-RAGE antibodies or fragments thereof described herein.

In another aspect, the disclosure concerns methods of inhibiting RAGE activity to prevent liver damage and/or fibrosis, said method comprising administering to the subject an effective amount of any of the anti-RAGE antibodies or fragments thereof described herein.

In another aspect, the disclosure concerns methods of inhibiting RAGE activity to prevent neovascularization in the eye, said method comprising administering to the subject an effective amount of any of the anti-RAGE antibodies or fragments thereof described herein.

In another aspect, the disclosure concerns methods of inhibiting RAGE activity to prevent respiratory diseases and/or the development of respiratory diseases, said method comprising administering to the subject an effective amount of any of the anti-RAGE antibodies or fragments thereof described herein.

In another aspect, the disclosure concerns methods of inhibiting RAGE activity to prevent infections and/or the development of infections, said method comprising administering to the subject an effective amount of any of the anti-RAGE antibodies or fragments thereof described herein.

In one aspect, the disclosure concerns methods of treating cancer, such as for example and not limitation, cancers characterized by high vascularization such as colorectal and renal cancers, said method comprising administering to the subject an effective amount of any of the anti-RAGE antibodies described herein.

In another aspect, methods of altering a tumor microenvironment, such as for example and not limitation, to inhibit vascularization to a tumor, inhibit growth of the tumor, inhibit or decrease expression of growth factors within the tumor, decrease or alter myeloid lineage cell type infiltration and/or activity, decrease or alter mesenchymal/stromal lineage cell type infiltration and/or activity, and/or to promote T cell activity and infiltration into the tumor, said method comprising administering to the subject an effective amount of any of the anti-RAGE antibodies or fragments thereof described herein.

In another aspect, the disclosure concerns methods of preventing the development of chronic inflammation, said method comprising administering to the subject an effective amount of any of the anti-RAGE antibodies or fragments thereof described herein. In a related aspect, the disclosure concerns maintaining an acute inflammatory state, said method comprising administering to the subject an effective amount of any of the anti-RAGE antibodies or fragments thereof described herein.

In another aspect, the disclosure concerns methods of preventing liver damage and/or fibrosis, said method comprising administering to the subject an effective amount of any of the anti-RAGE antibodies or fragments thereof described herein.

In another aspect, the disclosure concerns methods of preventing neovascularization in the eye, said method comprising administering to the subject an effective amount of any of the anti-RAGE antibodies or fragments thereof described herein.

In another aspect, the disclosure concerns methods of preventing respiratory diseases and/or the development of respiratory diseases, said method comprising administering to the subject an effective amount of any of the anti-RAGE antibodies or fragments thereof described herein.

In another aspect, the disclosure concerns methods of preventing infections and/or the development of infections, said method comprising administering to the subject an effective amount of any of the anti-RAGE antibodies or fragments thereof described herein.

In certain embodiments, the methods described herein further comprise administering to the subject an effective amount of a second medicament, wherein the anti-RAGE antibody or fragment thereof is the first medicament. In some embodiments, the second medicament is a vaccine, such as a vaccine to an infectious agent. In some embodiment, the second medicament is a biologic cancer therapeutic. Exemplary biological cancer therapeutics include, but not limited to, antibodies, cytokines, hematopoietic growth factors, cancer vaccines, bacillus Calmette-Guerin, an oncolytic virus, gene therapy, adoptive T-cell transfer therapy, chimeric antigen-receptor modified cells. Exemplary antibodies include, but are not limited to, those binding to PD-1, PD-L1, CTLA-4, GITIR, LAIR-1, TIGIT, CD73, OX40, TLR2, TLR4, TLR7, TLR8, TLR9, EP2 receptor, EP4 receptor, VEGF, CD20, CD52, EGF, and HER-2. Also included are small molecules that target for example, S100 family ligands, HBGB1, NFKB, TGF-BetalDO, TDO, ARG1, ARG2, iNOS, TLR2, TLR4, TLR7, TLR8, TLR9, PDE5, P2X7, P2Y11, A2A receptor, A2B receptor, CD39, CD73, COX2, EP2 receptor, EP4 receptor, CXCR1, CXCR2, CXCR4, CCR2, CCR5, ALK5, BRAF, RON, CSF1, PI3KGamma, PI3K delta, CSF receptor 1, and those also targeted by antibodies.

In certain embodiments, the subject or the individual is human.

In one aspect, the disclosure concerns a method of detecting RAGE protein in a sample suspected of containing the RAGE protein, the method comprising (a) contacting the sample with an anti-RAGE antibody or fragment thereof described herein; and (b) detecting formation of a complex between the anti-RAGE antibody or fragment thereof and the RAGE protein. In one embodiment, the anti-RAGE antibody or fragment thereof is detectably labeled.

Any embodiment described herein or any combination thereof applies to any and all anti-RAGE antibodies or fragments thereof, methods and uses of the disclosure described herein.

The antibodies or fragments thereof of the present application comprise a heavy chain constant region, such as an IgG1, IgG2, IgG3, IgG4, IgA, IgE, IgM or IgD constant region. Furthermore, the antibody can comprise a light chain constant region, either a kappa light chain constant region or a lambda light chain constant region. Particularly, the antibody comprises a kappa light chain constant region. Alternatively, the antibody portion can be, for example, a Fab fragment or a single chain Fv fragment. Replacements of amino acid residues in the Fc portion to alter antibody effector function are known in the art (U.S. Pat. Nos. 5,648,260; 5,624,821). The Fc portion of an antibody mediates several important effector functions. e.g. cytokine induction, ADCC, phagocytosis, complement dependent cytotoxicity (CDC) and half-life/clearance rate of antibody and antigen-antibody complexes. In some cases these effector functions are desirable for therapeutic antibodies but in other cases might be unnecessary or even deleterious, depending on the therapeutic objectives. Certain human IgG isotypes, particularly IgG1 and IgG3, mediate ADCC and CDC via binding to Fcγ Rs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

These and other objects, features and advantages of the present disclosure will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below. The patent or application file contains at least one drawing executed in color. Copies of this patent or patent application publication with color drawing(s) will be provided by the Office upon request and payment of the necessary fee.

FIGS. 1A-1B depicts binding and cross-reactivity of antibodies according to the disclosure by flow cytometry between human and mouse RAGE protein. FIG. 1A depicts binding to human RAGE protein. FIG. 1B depicts binding to mouse RAGE protein.

FIG. 2A-2C shows mapping of binding sites of antibodies of the disclosure to RAGE protein domains. FIG. 2A depicts a schematic of RAGE protein showing domains V, C1, and C2. FIG. 2B depicts the interaction of antibodies of the disclosure with RAGE domains V, C1, C2. FIG. 2C depicts a diagram showing binding sites of some exemplary antibodies of the disclosure relative to certain RAGE domains.

FIG. 3A-C shows the effects of anti-RAGE antibodies of the disclosure on various cell functions, including cell migration (FIG. 3A). Specifically, migration of THP-1 cells (human acute monocytic leukemia line) to AGE-BSA was determined. FIG. 3B depicts that anti-RAGE antibodies of the disclosure inhibit basal levels of NFkB signaling in a mouse melanoma cell line. FIG. 3C shows that antibodies of the disclosure block binding of S100A8/9 to monocytes and granulocytes.

FIG. 4 depicts the effects of anti-RAGE antibodies of the disclosure on Erk signaling.

FIG. 5A-5E shows binding of a specific anti-RAGE antibody, RFT01, in human colorectal cancer, adenocarcinoma cell line. FIG. 5A depicts binding of the RFT01 antibody to Stage III cells. FIG. 5B depicts binding of the RFT01 antibody to Stage IV cells. FIG. 5C depicts quantification of the RAGE antibody infiltrating cells in FIGS. 5A and 5B. FIG. 5D depicts binding of the RFT01 anti-RAGE antibody in human colorectal cancer, myeloid lineage cells. FIG. 5E depicts binding of the RFT01 anti-RAGE antibody in human colorectal cancer lymphoid cells.

FIG. 6A-B shows RS15 treatment effects on tumor infiltrating myeloid populations in syngeneic animal models CT26 and Colon26 (FIG. 6A) and the corresponding alteration in the tumoral cytokine profile in Colon26 tumors (FIG. 6B) toward a less immunosuppressive microenvironment.

FIG. 7 shows that anti-RAGE (RS15) treatment of mice with CT26 tumors increases tumor infiltrating T cells by FACS analysis of tumor infiltrating lymphoid populations.

FIG. 8A-8B shows that RFT01 treatment inhibits tumor growth. FIG. 8A shows that CT26 tumor growth is inhibited in a syngeneic mouse model (specifically mouse colon cancer cell line CT26 implanted into BALB/C mice). FIG. 8B shows that dosing of RFT01 starting with tumors of 100 mm3 resulted in a decrease in Colon26 tumor volume over time.

### DETAILED DESCRIPTION

### Definitions

To facilitate an understanding of the principles and features of the various embodiments of the disclosure, various illustrative embodiments are explained below. Although exemplary embodiments of the disclosure are explained in detail, it is to be understood that other embodiments are contemplated. Accordingly, it is not intended that the disclosure is limited in its scope to the details of construction and arrangement of components set forth in the following description or examples. The disclosure is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” or it may mean “both.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, up to ±10%, up to ±5%, or up to ±1% of a given value. Alternatively, particularly with respect to biological systems or processes, the term can mean within an order of magnitude, such as within two-fold, of a value. Where particular values are described in the application and claims, unless otherwise stated, the term “about” is implicit and in this context means within an acceptable error range for the particular value. In some embodiments, and unless otherwise indicated, the term “about” refers to values up to ±10% of a given value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claims or to imply that certain features are critical, essential, or even important to the structure or function of the claimed subject matter. Rather, these terms are merely intended to highlight alternative or additional features that may or may not be utilized in a particular embodiment of the present disclosure. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present disclosure are intended to be illustrative and not restrictive. Many suitable materials that would perform the same or a similar function as the materials described herein are intended to be embraced within the scope of the disclosure. Such other materials not described herein can include, but are not limited to, materials that are developed later in time, for example. Any dimensions listed in the various drawings are for illustrative purposes only and are not intended to be limiting. Other dimensions and proportions are contemplated and intended to be included within the scope of the disclosure.

As used herein, the term “subject” or “patient” or “individual” refers to mammals and includes, without limitation, domestic animals (e.g., cows, sheep, cats, dogs, and horses), primates (e.g., humans and non-human primates such as monkeys), rabbits, and rodents (e.g., mice and rats). In certain embodiment, the subject is human.

As used herein, the term “sample” refers to anything which may contain an analyte for which an analyte assay is desired. The sample may be a biological sample, such as a biological fluid or a biological tissue. Examples of biological fluids include blood, serum, plasma, saliva, sputum, ocular lens fluid, sweat, urine, milk, ascites fluid, mucous, synovial fluid, peritoneal fluid, transdermal exudates, pharyngeal exudates, bronchoalveolar lavage, tracheal aspirations, cerebrospinal fluid, semen, cervical mucus, vaginal or urethral secretions, amniotic fluid, and the like. Biological tissues comprise an aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample can be used as obtained directly from the source or following a pretreatment so as to modify its character.

As used herein, the term “specifically binds” refers to the binding specificity of a specific binding pair. Recognition by an antibody of a particular target in the presence of other potential targets is one characteristic of such binding.

As used herein, the term “combination” of an anti-RAGE antibody and at least a second pharmaceutically active ingredient means at least two, but any desired combination of compounds can be delivered simultaneously or sequentially (e.g., within a 24 hour period). It is contemplated that when used to treat various diseases, the compositions and methods of the present disclosure can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

Within the meaning of the present disclosure, the term “conjoint administration” is used to refer to administration of a composition according to the disclosure and another therapeutic agent simultaneously in one composition, or simultaneously in different compositions, or sequentially (such as within a 24 hour period).

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein the term “therapeutically effective” or “effective” applied to dose or amount refers to that quantity of a compound or pharmaceutical composition that when administered to a subject for treating (e.g., preventing or ameliorating) a state, disorder or condition, is sufficient to effect such treatment or prophylactic result. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the disclosure, refers to molecular entities and other ingredients of such compositions that are physiologically tolerable and do not typically produce untoward reactions when administered to a mammal (e.g., a human). In some embodiments, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The terms “anti-RAGE antibody”, “anti-RAGE”, “RAGE antibody” or “an antibody that binds to RAGE” refers to an antibody that is capable of binding RAGE (e.g., human RAGE having an amino acid sequence set forth in SEQ ID NO: 1) with sufficient affinity such that the antibody is useful as a diagnostic and/or therapeutic agent in targeting RAGE. In one embodiment, the extent of binding of an anti-RAGE antibody to an unrelated, non-RAGE protein is less than about 10% of the binding of the antibody to RAGE as measured, e.g., by a radioimmunoassay (RIA). In certain embodiments, an antibody that binds to PCSK9 has a dissociation constant (Kd) of <1 μM, <100 nM, <10 nM, <1 nM, <0.1 nM, <0.01 nM, or <0.001 nM (e.g. 10⁻⁸M or less, e.g. from 10⁻⁸ M to 10⁻¹³ M, e.g., from 10⁻¹ M to 10⁻¹³ M). In certain embodiments, an anti-RAGE antibody binds to an epitope of RAGE that is conserved among RAGE from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art, nonlimiting embodiments of which are discussed below. An antibody is said to be “capable of binding” a molecule if it is capable of specifically reacting with the molecule to thereby bind the molecule to the antibody. As used herein, the term “fragment”, when referring to an antibody should be read to mean an antigen-binding fragment, such as a RAGE-binding antibody fragment.

An “antigen-binding portion” or “antigen-binding fragment” of an antibody (or simply “antibody portion” or “antibody fragment”) refers to a molecule other than an intact antibody that comprises a portion of an intact antibody that binds the antigen to which the intact antibody binds (e.g., one or more fragments of an antibody that retain the ability to specifically bind to an antigen). Examples of antibody fragments include but are not limited to Fv, Fab, Fab′, Fab′-SH, F(ab′)₂, diabodies; linear antibodies, single-chain antibody molecules (e.g. scFv), heavy chain only antibodies (HCAb), and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. Other forms of single chain antibodies, such as diabodies are also encompassed.

The term “chimeric” antibody refers to an antibody in which a portion of the heavy and/or light chain is derived from a particular source or species, and at least one other portion of the heavy and/or light chain, including the remainder thereof, is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁; IgG₂, IgG₃, IgG₄, IgA₁; and IgA₂. The heavy chain constant domains that correspond to the different classes of immunoglobulins are called α, δ, ε, γ, and μ, respectively.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent and/or bispecific. Diabodies are described more fully in, for example, EP404,097; WO1993/01161; Hudson et al., Nat. Med. 9:129-134, 2003; and Hollinger et al., Proc. Natl. Acad. Sci. USA 90:6444-6448, 1993. Triabodies and tetrabodies are also described in Hudson et al., Nat. Med. 9:129-134. 2003.

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The “Fab” fragment contains the heavy- and light-chain variable domains and also contains the constant domain of the light chain and the first constant domain (CHI) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions.

“Framework” or “FR” refers to variable domain residues other than complementarity determining region (CDR) residues. The FR of a variable domain generally consists of four FR domains: FR1, FR2, FR3, and FR4. Accordingly, the CDR and FR sequences generally appear in the following sequence in VH (or VL): FR1-CDRH1(CDRL1)-FR2-CDRH2(CDRL2)-FR3-CDRH3(CDRL3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

A “humanized” antibody refers to an antibody comprising heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. A humanized antibody may thus comprise amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. Particularly, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain. The humanized antibody can be selected from any class of immunoglobulins, including IgY, IgM, IgG, IgD, IgA and IgE, and any isotype, including without limitation IgA1, IgA2, IgG1, IgG2, IgG3 and IgG4. The humanized antibody may comprise sequences from more than one class or isotype, and particular constant domains may be selected to optimize desired effector functions using techniques well-known in the art. The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a particular embodiment, such mutations, however, will not be extensive. Usually, at least 50, 55, 60, 65, 70, 75 or 80%, particularly at least 85%, more particularly at least 90%, and in particular at least 95% of the humanized antibody residues will correspond to those of the parental FR and CDR sequences. Thus, disclosed herein are humanized antibodies in which at least one of the CDRs correspond to one of SEQ ID Nos:4-9 and 12-17.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

An “immunoconjugate” is an antibody conjugated to one or more heterologous molecule(s), including but not limited to a cytotoxic agent.

An “isolated” antibody is one which has been separated from a component of its natural environment. In some embodiments, an antibody is purified to greater than 95% or 99% purity as determined by, for example and not limitation, electrophoretic methods (e.g., SDS-PAGE, isoelectric focusing (IEF), capillary electrophoresis) or chromatographic methods (e.g., ion exchange or reverse phase HPLC). An isolated antibody is substantially free of other antibodies having different antigenic specificities (e.g., an isolated antibody that specifically binds human RAGE is substantially free of antibodies that specifically bind antigens other than human RAGE). An isolated antibody that specifically binds human RAGE may, however, have cross-reactivity to other antigens, such as RAGE molecules from other species

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-RAGE antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “polynucleotide” as referred to herein means a polymeric form of two or more nucleotides, either ribonucleotides or deoxynucleotides or a modified form of either type of nucleotide. The term includes single and double stranded forms of DNA but particularly is double-stranded DNA.

The term “isolated polynucleotide” as used herein shall mean a polynucleotide (e.g., of genomic, cDNA, or synthetic origin, or some combination thereof) that, by virtue of its origin, the “isolated polynucleotide”: is not associated with all or a portion of a polynucleotide with which the “isolated polynucleotide” is found in nature; is operably linked to a polynucleotide that it is not linked to in nature; or does not occur in nature as part of a larger sequence.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions termed complementarity determining regions (CDRs). (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds.(1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Compositions and Methods

The structure of human RAGE has been determined in the ligand-bound form (to human S100A6) and in the unbound form. RAGE has an extracellular portion, a transmembrane domain, and a short cytoplasmic portion. Homology to other proteins led to a model where RAGE has several extracellular domains. These extracellular domains are named in analogy to immunoglobulins: V domain, C1 domain, and C2 domain (so named for their homology to the V and C domains of immunoglobulins). The V domain lies at the N-terminus and binds to some ligands like S100 (specifically S100B, S100A1, S100A2, S100A4, S100A5, S100A6, S100A7, S100A8/9, S100A11, S100A12, S100A13, S100P) and AGEs. A monoclonal antibody binding to the V-like domain in RAGE competes with binding of different ligands including, but not limited to, S100b, HMGB1, and amyloid p implying that these ligands would also bind to RAGE via this same domain. Two domains within RAGE have homology to the C2 domains of immunoglobulins; one of these domains is called C1. Several ligands binding to this domain have been described, for instance S100A12 (also called ENRAGE or Calgranulin C) and amyloid-β-peptide aggregates. The second, C2-like, domain is called C2. RAGE ligand S100A6 binds to the C2 domain. Human RAGE also has a signal peptide. Generally, the overall structure of human RAGE is considered to be a signal peptide (amino acids 1-22 of SEQ ID NO: 1; also shown in SEQ ID NO: 21), followed by three immunoglobulin-like domains, including an Ig-like V-type domain (amino acids 23-116 of SEQ ID NO: 1; also shown in SEQ ID NO: 18) and two Ig-like C2-type 1/2 domains (amino acids 124-221 of SEQ ID NO: 1; also designated C1 domain and shown in SEQ ID NO: 19; and amino acids 227-317 of SEQ ID NO: 1; also designated C2 domain and shown in SEQ ID NO: 20); a single transmembrane domain (amino acids 343-363 of SEQ ID NO: 1), and a short cytoplasmic tail (amino acids 364-404 of SEQ ID NO: 1; also shown in SEQ ID NO: 22). Human RAGE includes both the membrane-bound form and a soluble form derived from the V, C1, and C2 extracellular domains.

RAGE is a target for other indications where pathologies of disease are inflammatory-driven, causing tissue dysfunction via inflammatory priming, amplification, and neovascularization. Examples of these indications include atherosclerosis, psoriasis, diabetic retinopathy, diabetic neuropathy, nephropathy, and Alzheimer's disease.

RAGE polymorphisms have also been linked to several disease indications. Autoimmune indications include, such as for example and not limitation, Type 1 diabetes mellitus, psoriasis, and rheumatoid arthritis. Vascular disease indications include, such as for example and not limitation, atherosclerosis, sporadic abdominal aortic aneurysms, and diabetic microvascular dermatoses. Oncology indications include, such as for example and not limitation, gastric cancers.

RAGE expression correlates with poor prognosis and survival, stage of disease, and metastasis in human tumors. Specifically, higher RAGE expression is associated with poor clinical outcome in the literature for certain cancer types (such as, but not limited to, gastric cancer, breast cancer, and renal cancer).

Experiments have been performed to determine the effects of knocking out RAGE in various animal models. For example, it has been shown that RAGE-knockout mice are resistant to CAC (colitis-associated carcinogenesis). RAGE-knockout mice are resistant to DMBA/TPA-induced skin carcinogenesis, which is a common model of chemical-induced skin carcinogenesis using 7, 12-dimethylbenzanthracene (DMBA) and 12-O-tetradecanoylphorbol-13-acetate (TPA) as tumor initiator and promoter, respectively. RAGE-knockout mice also show delayed mutant Ras-promoted pancreatic carcinogenesis. RAGE-knockout mice show decreased growth of orthotopic polyoma middle T oncoprotein (PyMT) breast tumors. Others have shown that RAGE-knockout mice have longer survival when implanted orthotopically with a GL261 glioma cell line, possibly as a result of decreased tumor inflammation and vascularization mediated by TAMs and microglia. Still others have shown that tumor suppressor antigen presenting cell (APC) mutant mice have decreased intestinal tumor incidence and size in RAGE-knockout mice controls. Further, a Kras mutant transgenic model of pancreatic ductal carcinoma displayed carcinogenesis when crossed with RAGE knockout mice. In an orthotopic syngeneic breast cancer model, transgenic mice over expressing RAGE ligand S100a7a15, were treated with either soluble RAGE or a RAGE neutralizing antibody. The resulting RAGE inhibition decreased tumor growth, lung metastasis, vascularization (CD31), and TAM recruitment (f4/80).

In a syngeneic animal model, RAGE-transfected MC-38 tumor cells injected into the portal vein caused increased tumor burden compared to controls. In another syngeneic animal model, CT26 tumor cells were injected into the portal vein and treated with soluble RAGE. This resulted in reduced tumor burden in the liver.

RAGE inhibition is also effective in xenograft models, suggesting non-immune mediated mechanisms. For example, siRAGE-transfected MDA-MB-231 cells implanted in the mammary fat pad of NOD/SCID which were then injected intratumorally with S100A8/9 showed less metastasis to the lungs. Single progeny clone 2 of MBA-MD-231 injected intracardially into nude mice showed significantly less metastatic potential when treated with RAGE neutralizing antibody.

In one aspect, the disclosure is based, in part, on experimental and clinical results obtained with anti-RAGE antibodies. Results obtained indicate that anti-RAGE antibodies of the disclosure are capable of inhibiting Erk/MAPK signaling, inhibiting motility and invasiveness of cancer cells, promoting T cell infiltration into tumor cells, altering tumor microenvironments, and inhibiting tumor growth.

### A. Exemplary Anti-RAGE Antibodies

In one aspect, the disclosure provides isolated antibodies, or fragments thereof, that bind to RAGE, such as the RAGE human isoform. In certain embodiments, an anti-RAGE antibody, or fragment thereof, modulates RAGE activity. Any of the antibodies, or fragments thereof, described herein can specifically bind to RAGE, including, but not limited to, particular domains of RAGE as discussed herein, as well as epitopes of RAGE.

In certain embodiments, an anti-RAGE antibody or fragment thereof binds to at least one domain of RAGE, such as for example and not limitation, the V and/or C1 domains of human RAGE. In certain embodiments, an anti-RAGE antibody or fragment thereof binds to at least one epitope of RAGE, including for example and not limitation, an epitope found in the V and/or C1 domains of human RAGE.

In certain embodiments, an anti-RAGE antibody or fragment thereof binds to an amino acid sequence that is at least 90%, 91%, 92%, 93%, 94%, 95%, 96%, 97%, 98%, 99%, or 100% identical to at least a portion of SEQ ID NO: 1. This portion may be contained in any of, at least one of, at least two of, or all of the V, C1, and/or C2 domains of human RAGE. In certain embodiments, an anti-RAGE antibody or fragment thereof binds to an amino acid sequence that is at least 90%, 91%, 92%, 93%, 94%, 95%, 96%, 97%, 98%, 99%, or 100% identical to at least a portion of the V and/or C1 domains of human RAGE. In certain embodiments, an anti-RAGE antibody or fragment thereof binds to an amino acid sequence that is at least 90%, 91%, 92%, 93%, 94%, 95%, 96%, 97%, 98%, 99%, or 100% identical to at least a portion of SEQ ID NOs: 18, 19, and/or 20.

In one aspect, the disclosure provides an antibody or an antibody fragment that binds to RAGE (e.g., SEQ ID NO: 1) or a fragment thereof (including homologous variants of RAGE and a fragment thereof), wherein the antibody or fragment thereof comprises a variable domain comprising a heavy chain (VH) comprising the amino acid sequence of SEQ ID NO: 2 or 10 and a light chain (VL) comprising the amino acid sequence of SEQ ID NO: 3 or 11. In certain embodiments, the antibody or fragment thereof comprises a VH domain comprising an amino acid sequence that is at least 90%, 91%, 92%, 93%, 94%, 95%, 96%, 97%, 98%, 99%, or 100% identical to SEQ ID NO: 2 or 10. In certain embodiments, the antibody or fragment thereof comprises a variable domain comprising a VL domain comprising an amino acid sequence that is at least 90%, 91%, 92%, 93%, 94%, 95%, 96%, 97%, 98%, 99%, or 100% identical to SEQ ID NO: 3 or 11. In certain embodiments, the sequence identify is to a full-length sequence disclosed herein.

In one embodiment, an antibody or an antibody fragment that binds to RAGE or a fragment thereof (including homologous variants of RAGE and a fragment thereof) is provided, wherein the antibody or fragment thereof comprises a VH comprising the amino acid sequence of SEQ ID NO: 2. In certain embodiments, the antibody or fragment thereof further comprises a VL comprising the amino acid sequence of SEQ ID NO: 3. In certain embodiments, the antibody or fragment thereof comprises a VH comprising the amino acid sequence of SEQ ID NO: 2 and a VL comprising the amino acid sequence of SEQ ID NO: 3. In certain embodiments, the antibody or fragment thereof comprises a VH domain comprising an amino acid sequence that is at least 90%, 91%, 92%, 93%, 94%, 95%, 96%, 97%, 98%, 99%, or 100% identical to SEQ ID NO: 2. In certain embodiments, the antibody or fragment thereof comprises a VL domain comprising an amino acid sequence that is at least 90%, 91%, 92%, 93%, 94%, 95%, 96%, 97%, 98%, 99%, or 100% identical to SEQ ID NO: 3. In certain embodiments, the sequence identify is to a full-length sequence disclosed herein.

In one embodiment, an antibody or an antibody fragment that binds to RAGE or a fragment thereof (including homologous variants of RAGE and a fragment thereof) is provided, wherein the antibody or fragment thereof comprises a VH comprising the amino acid sequence of SEQ ID NO: 10. In certain embodiments, the antibody or fragment thereof further comprises a VL comprising the amino acid sequence of SEQ ID NO: 11. In certain embodiments, the antibody or fragment thereof comprises a VH comprising the amino acid sequence of SEQ ID NO: 10 and a VL comprising the amino acid sequence of SEQ ID NO: 11. In certain embodiments, the antibody or fragment thereof comprises a variable domain comprising a heavy chain (VH) comprising an amino acid sequence that is at least 90%, 91%, 92%, 93%, 94%, 95%, 96%, 97%, 98%, 99%, or 100% identical to SEQ ID NO: 10. In certain embodiments, the antibody or fragment thereof comprises a variable domain comprising a light chain (VL) comprising an amino acid sequence that is at least 90%, 91%, 92%, 93%, 94%, 95%, 96%, 97%, 98%, 99%, or 100% identical to SEQ ID NO: 11.

In certain embodiments, an antibody or an antibody fragment that binds to RAGE or a fragment thereof (including homologous variants of RAGE and a fragment thereof) is provided, wherein the antibody or fragment thereof comprises a VH sequence having at least 90%, 91%, 92%, 93%, 94%, 95%, 96%, 97%, 98%, 99%, or 100% sequence identity to the amino acid sequence of SEQ ID NO: 2, or SEQ ID NO: 10. In certain embodiments, a VH sequence having at least 90%, 91%, 92%, 93%, 94%, 95%, 96%, 97%, 98%, 99%, or 100% identity contains substitutions (e.g., conservative substitutions), insertions, or deletions relative to the reference sequence, but an anti-RAGE antibody or fragment thereof comprising that sequence retains the ability to bind to RAGE (e.g., SEQ ID NO: 1).

In certain embodiments, an antibody or an antibody fragment that binds to RAGE or a fragment thereof (including homologous variants of RAGE and a fragment thereof) is provided, wherein the antibody or fragment thereof comprises a VL sequence having at least 90%, 91%, 92%, 93%, 94%, 95%, 96%, 97%, 98%, 99%, or 100% sequence identity to the amino acid sequence of SEQ ID NO: 3, or SEQ ID NO: 11. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-RAGE antibody comprising that sequence retains the ability to bind to RAGE (e.g., SEQ ID NO: 1).

In one embodiment, an antibody or an antibody fragment that binds to RAGE or a fragment thereof (including homologous variants of RAGE and a fragment thereof) is provided, wherein the antibody or fragment thereof comprises a VH sequence of SEQ ID NO: 2 and a VL sequence of SEQ ID NO: 3. In another embodiment, an antibody or an antibody fragment that binds to RAGE or a fragment thereof is provided, wherein the antibody comprises a VH sequence of SEQ ID NO: 10 and a VL sequence of SEQ ID NO: 11.

In certain embodiments, an antibody or an antibody fragment that binds to RAGE or a fragment thereof (including homologous variants of RAGE and a fragment thereof) is provided, wherein the antibody or fragment thereof comprises a variable domain comprising at least one, two, three, four, five or six complementarity determining region (CDR) sequences comprising: CDR-H1 comprising the amino acid sequence of SEQ ID NO: 4 or 12; CDR-H2 comprising the amino acid sequence of SEQ ID NO: 5 or 13; and CDR-H3 comprising the amino acid sequence of SEQ ID NO: 6 or 14. In certain embodiments, the antibody or fragment thereof further comprises CDR-L1 comprising the amino acid sequence of SEQ ID NO: 7 or 15: CDR-L2 comprising the amino acid sequence of SEQ ID NO: 8 or 16; and CDR-L3 comprising the amino acid sequence of SEQ ID NO: 9 or 17. In certain embodiments, the antibody or fragment thereof comprises a variable domain comprising at least one, two, three, four, five or CDR sequences comprising amino acid sequences that are at least 90%, 91%, 92%, 93%, 94%, 95%, 96%, 97%, 98%, 99%, or 100% identical to any of SEQ ID NOs 4-9 or 12-17.

In certain embodiments, an antibody or an antibody fragment that binds to RAGE or a fragment thereof (including homologous variants of RAGE and a fragment thereof) is provided, wherein the antibody or fragment thereof comprises a variable domain comprising six CDR sequences comprising: CDR-H1 comprising the amino acid sequence of SEQ ID NO: 4 or 12; CDR-H2 comprising the amino acid sequence of SEQ ID NO: 5 or 13; and CDR-H3 comprising the amino acid sequence of SEQ ID NO: 6 or 14. In certain embodiments, the antibody or fragment thereof further comprises CDR-L1 comprising the amino acid sequence of SEQ ID NO: 7 or 15: CDR-L2 comprising the amino acid sequence of SEQ ID NO: 8 or 16; and CDR-L3 comprising the amino acid sequence of SEQ ID NO: 9 or 17. In certain embodiments, the antibody or fragment thereof comprises a variable domain comprising six CDR sequences comprising amino acid sequences that are at least 90%, 91%, 92%, 93%, 94%, 95%, 96%, 97%, 98%, 99%, or 100% identical to any of SEQ ID NOs 4-9 or 12-17.

In one aspect, the disclosure provides an antibody or an antibody fragment that binds to RAGE or a fragment thereof (including homologous variants of RAGE and a fragment thereof) comprising at least one, at least two, or all three VH CDR sequences selected from (a) CDR-H1 comprising the amino acid sequence of SEQ ID NO: 4, or SEQ ID NO: 12; (b) CDR-H2 comprising the amino acid sequence of SEQ ID NO: 5 or SEQ ID NO: 13; and (c) CDR-H3 comprising the amino acid sequence of SEQ ID NO: 6 or SEQ ID NO: 14. In certain embodiments, the antibody or fragment thereof comprises a variable domain comprising at least one, at least two, or all three VH CDR sequences comprising amino acid sequences that are at least 90%, 91%, 92%, 93%, 94%, 95%, 96%, 97%, 98%, 99%, or 100% identical to any of SEQ ID NOs 4-6 or 12-14.

In one aspect, the disclosure provides an antibody or an antibody fragment that binds to RAGE or a fragment thereof (including homologous variants of RAGE and a fragment thereof) comprising at least one, at least two, or all three VL CDR sequences selected from (a) CDR-L1 comprising the amino acid sequence of SEQ ID NO: 7, or SEQ ID NO: 15; (b) CDR-L2 comprising the amino acid sequence of SEQ ID NO: 8 or SEQ ID NO: 16; and (c) CDR-L3 comprising the amino acid sequence of SEQ ID NO: 9 or SEQ ID NO: 17. In certain embodiments, the antibody or fragment thereof comprises a variable domain comprising at least one, at least two, or all three VL CDR sequences comprising amino acid sequences that are at least 90%, 91%, 92%, 93%, 94%, 95%, 96%, 97%, 98%, 99%, or 100% identical to any of SEQ ID NOs 7-9 or 15-17.

In another aspect, the disclosure provides an antibody or an antibody fragment that binds to RAGE or a fragment thereof (including homologous variants of RAGE and a fragment thereof) comprising (a) a VH domain comprising at least one, at least two, or all three VH CDR sequences selected from (i) CDR-H1 comprising the amino acid sequence of SEQ ID NO: 4, or SEQ ID NO: 12; (ii) CDR-H2 comprising the amino acid sequence of SEQ ID NO: 5 or SEQ ID NO: 13; and (iii) CDR-H3 comprising the amino acid sequence of SEQ ID NO: 6 or SEQ ID NO: 14; and (b) a VL domain comprising at least one, at least two, or all three VL CDR sequences selected from (i) CDR-L1 comprising the amino acid sequence of SEQ ID NO: 7, or SEQ ID NO: 15; (ii) CDR-L2 comprising the amino acid sequence of SEQ ID NO: 8 or SEQ ID NO: 16; and (iii) CDR-L3 comprising the amino acid sequence of SEQ ID NO: 9 or SEQ ID NO: 17. In certain embodiments, the antibody or fragment thereof comprises a variable domain comprising at least one, at least two, or all three VH and VL CDR sequences comprising amino acid sequences that are at least 90%, 91%, 92%, 93%, 94%, 95%, 96%, 97%, 98%, 99%, or 100% identical to any of SEQ ID NOs 4-9 or 12-17.

In certain embodiments, an antibody or an antibody fragment that binds to RAGE or a fragment thereof (including homologous variants of RAGE and a fragment thereof) is provided, wherein the antibody or fragment thereof comprises a VH sequence comprising at least one, at least two, or all three VH CDR sequences selected from (i) a CDR-H1 having at least 90%, 91%, 92%, 93%, 94%, 95%, 96%, 97%, 98%, 99%, or 100% sequence identity to the amino acid sequence of SEQ ID NO: 4, or SEQ ID NO: 12; (ii) a CDR-H2 having at least 90%, 91%, 92%, 93%, 94%, 95%, 96%, 97%, 98%, 99%, or 100% sequence identity to the amino acid sequence of SEQ ID NO: 5 or SEQ ID NO: 13; and (iii) a CDR-H3 having at least 90%, 91%, 92%, 93%, 94%, 95%, 96%, 97%, 98%, 99%, or 100% sequence identity to the amino acid sequence of SEQ ID NO: 6 or SEQ ID NO: 14. In certain embodiments, a CDR-H1, CDR-H2 or CDR-H3 sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-RAGE antibody or fragment thereof comprising that sequence retains the ability to bind to RAGE (e.g., SEQ ID NO: 1).

In certain embodiments, an antibody or an antibody fragment that binds to RAGE or a fragment thereof (including homologous variants of RAGE and a fragment thereof) is provided, wherein the antibody or fragment thereof comprises a VL sequence comprising at least one, at least two, or all three VL CDR sequences selected from (i) a CDR-L1 having at least 90%, 91%, 92%, 93%, 94%, 95%, 96%, 97%, 98%, 99%, or 100% sequence identity to the amino acid sequence of SEQ ID NO: 7, or SEQ ID NO: 15; (ii) a CDR-L2 having at least 90%, 91%, 92%, 93%, 94%, 95%, 96%, 97%, 98%, 99%, or 100% sequence identity to the amino acid sequence of SEQ ID NO: 8 or SEQ ID NO: 16; and (iii) a CDR-L3 having at least 90%, 91%, 92%, 93%, 94%, 95%, 96%, 97%, 98%, 99%, or 100% sequence identity to the amino acid sequence of SEQ ID NO: 9 or SEQ ID NO: 17. In certain embodiments, a CDR-L1, CDR-L2 or CDR-L3 sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-RAGE antibody or fragment thereof comprising that sequence retains the ability to bind to RAGE (e.g., SEQ ID NO: 1).

In one embodiment, an antibody or an antibody fragment that binds to RAGE or a fragment thereof (including homologous variants of RAGE and a fragment thereof) is provided, wherein the antibody or fragment thereof comprises:
(1) a CDR-H1 comprising the amino acid sequence of SEQ ID NO: 4;
(2) a CDR-H2 comprising the amino acid sequence of SEQ ID NO: 5;
(3) a CDR-H3 comprising the amino acid sequence of SEQ ID NO: 6;
(4) a CDR-L1 comprising the amino acid sequence of SEQ ID NO: 7;
(5) a CDR-L2 comprising the amino acid sequence of SEQ ID NO: 8; and
(6) a CDR-L3 comprising the amino acid sequence of SEQ ID NO: 9.

In certain embodiments, the antibody or antibody fragment comprises amino acid sequences that are at least 90%, 91%, 92%, 93%, 94%, 95%, 96%, 97%, 98%, 99%, or 100% identical to any of SEQ ID NOs 4-9.

In one embodiment, an antibody or an antibody fragment that binds to RAGE or a fragment thereof (including homologous variants of RAGE and a fragment thereof) is provided, wherein the antibody or fragment thereof comprises:
(1) a CDR-H1 comprising the amino acid sequence of SEQ ID NO: 12;
(2) a CDR-H2 comprising the amino acid sequence of SEQ ID NO: 13;
(3) a CDR-H3 comprising the amino acid sequence of SEQ ID NO: 14;
(4) a CDR-L1 comprising the amino acid sequence of SEQ ID NO: 15;
(5) a CDR-L2 comprising the amino acid sequence of SEQ ID NO: 16; and
(6) a CDR-L3 comprising the amino acid sequence of SEQ ID NO: 17.

In certain embodiments, the antibody or antibody fragment comprises amino acid sequences that are at least 90%, 91%, 92%, 93%, 94%, 95%, 96%, 97%, 98%, 99%, or 100% identical to any of SEQ ID NOs 12-17.

In one embodiment, an antibody or an antibody fragment that binds to RAGE or a fragment thereof (including homologous variants of RAGE and a fragment thereof) is provided, wherein the antibody or fragment thereof comprises at least one CDR comprising an amino acid sequence selected from the group consisting of: SEQ ID NO: 4, SEQ ID NO: 5, SEQ ID NO: 6, SEQ ID NO: 7, SEQ ID NO: 8, SEQ ID NO: 9, SEQ ID NO: 12, SEQ ID NO: 13, SEQ ID NO: 14, SEQ ID NO: 15, SEQ ID NO: 16, and SEQ ID NO: 17.

In one embodiment, an antibody or an antibody fragment that binds to RAGE or a fragment thereof (including homologous variants of RAGE and a fragment thereof) is provided, wherein the antibody or fragment thereof comprises at least one CDR having at least 90%, 91%, 92%, 93%, 94%, 95%, 96%, 97%, 98%, 99%, or 100% sequence identity to an amino acid sequence selected from the group consisting of: SEQ ID NO: 4, SEQ ID NO: 5, SEQ ID NO: 6, SEQ ID NO: 7, SEQ ID NO: 8, SEQ ID NO: 9, SEQ ID NO: 12, SEQ ID NO: 13, SEQ ID NO: 14, SEQ ID NO: 15, SEQ ID NO: 16, and SEQ ID NO: 17.

In another aspect, an anti-RAGE antibody or fragment thereof is provided, wherein the antibody or fragment comprises a VH as in any of the embodiments provided above, and a VL as in any of the embodiments provided above.

In another aspect, an anti-RAGE antibody or fragment thereof is provided, wherein the antibody or fragment comprises any CDR as in any of the embodiments above. In another aspect, an anti-RAGE antibody or fragment thereof is provided, wherein the antibody or fragment comprises any CDR-H1, CDR-H2, or CDR-H3 as in any of the embodiments provided above, and any CDR-L1, CDR-L2, or CDR-L3 as in any of the embodiments provided above.

In another aspect, the anti-RAGE antibody or fragment thereof includes post-translational modifications.

In certain embodiments, an antibody or an antibody fragment that binds to RAGE or a fragment thereof is provided, wherein the antibody or fragment thereof binds to an epitope within a fragment of RAGE. In certain embodiments, an antibody or an antibody fragment that binds to RAGE or a fragment thereof is provided, wherein the antibody binds to an epitope within a fragment of RAGE comprising the V and/or C1 domains of the human RAGE amino acid sequence of SEQ ID NO: 1. In certain embodiments, an antibody or fragment thereof binds to an epitope that has at least 90%, 91%, 92%, 93%, 94%, 95%, 96%, 97, 98%, 99%, or 100% identity to RAGE and/or a fragment thereof, including but not limited to a fragment comprising the V and/or C1 domains of human RAGE.

In certain embodiments, the anti-RAGE antibody is a monoclonal antibody. In certain embodiments, the anti-RAGE antibody is a chimeric antibody. In certain embodiments, the anti-RAGE antibody is a humanized antibody. In certain embodiments, the anti-RAGE antibody is a human antibody. In certain embodiments, at least a portion of the framework sequence of the anti-RAGE antibody is a human consensus framework sequence. In one embodiment, the antibody is an RAGE-binding antibody fragment selected from a Fab, Fab′-SH, Fv, scFv, or (Fab′)₂ fragment. In some embodiments, a RAGE-binding antibody, or fragment thereof, disclosed herein is any antibody, or variant thereof, containing one or more CDRs selected from SEQ ID Nos. 4-9 and 12-17 and which binds to human RAGE.

In any of the above embodiments, the anti-RAGE antibody, or fragment thereof, is capable of binding a target selected from RAGE molecules. In any of the above embodiments, the anti-RAGE antibody or fragment thereof is capable of binding to human RAGE. In any of the above embodiments, the anti-RAGE antibody or fragment thereof is capable of binding to mouse or rat RAGE.

In any of the above embodiments, the anti-RAGE antibody or fragment thereof is capable of modulating, in particular neutralizing, a biological function of a target, selected from RAGE molecules. In any of the above embodiments, the anti-RAGE antibody or fragment thereof modulates, in particular inhibits, the ability of RAGE to bind to at least one of its ligands.

In any of the above embodiments, the anti-RAGE antibody or fragment thereof comprises a linker polypeptide or an immunoglobulin constant domain.

In any of the above embodiments, the anti-RAGE antibody or fragment thereof is selected from the group consisting of an immunoglobulin molecule, a monoclonal antibody, a chimeric antibody, a CDR-grafted antibody, a humanized antibody, a Fab, a Fab′, a F(ab′)₂, a Fv, a disulfide linked Fv, a scFv, a single domain antibody, a diabody, a multispecific antibody, a dual specific antibody, a dual variable domain immunoglobulin, a heavy chain antibody (HCAb), and a bispecific antibody.

In any of the above embodiments, the anti-RAGE antibody or fragment thereof comprises a heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, a human IgD constant domain, a human IgA1 constant domain, a human IgA2 constant domain, a human IgY constant domain, and corresponding mutated domains.

In any of the above embodiments, the anti-RAGE antibody or fragment thereof is present as an immunoconjugate, further comprising an agent selected from the group consisting of an immunoadhesion molecule, an imaging agent (such as for example and not limitation, a radiolabel (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm), an enzyme, a fluorescent label, a luminescent label, a bioluminescent label, a magnetic label, and biotin), a therapeutic agent, and a cytotoxic agent. Exemplary therapeutic or cytotoxic agents include an anti-metabolite, an alkylating agent, an antibiotic, a growth factor, a cytokine, an anti-angiogenic agent, an anti-mitotic agent, an anthracycline, toxin, and an apoptotic agent.

In one aspect, the disclosure provides an isolated CDR of an antibody as described herein.

In another aspect, the disclosure provides an isolated binding protein that specifically interacts with at least one epitope of a RAGE protein and/or a homologous variant of that epitope that has at least 90%, 91%, 92%, 93%, 94%, 95%, 96%, 97, 98%, 99%, or 100% identity to RAGE. In a related aspect, the isolated binding protein is an antibody or an antigen-binding fragment thereof. In certain embodiments, the antibody, or an antigen-binding fragment thereof, comprises a VH and a VL domain and/or at least one CDR.

In one aspect, a nucleic acid encoding any of the above anti-RAGE antibodies or fragments thereof is provided. In one embodiment, a vector comprising the nucleic acid is provided. In one embodiment, the vector is an expression vector. In one embodiment, a host cell comprising the vector is provided. In another embodiment, a host cell transformed with the vector is provided. In one embodiment, the host cell is eukaryotic (such as for example and not limitation, protist cell, animal cell, plant cell, fungal cell, mammalian cell, avian cell, insect cell, HEK Cells, CHO cells, COS cells and yeast cells). In another embodiment, the host cell is mammalian. In yet another embodiment, the host cell is prokaryotic.

In one aspect, the disclosure provides a hybridoma cell line that produces an anti-RAGE antibody or antigen-binding fragment thereof. In certain embodiments, the hybridoma is selected from the group consisting of mouse, human, rat, sheep, pig, cattle, goat, and horse hybridoma. In certain embodiments, the hybridoma cell line produces an anti-RAGE antibody or antigen-binding fragment thereof (e.g., a monoclonal antibody), which specifically binds to at least one epitope of a RAGE protein.

In one embodiment, a method of making an anti-RAGE antibody or fragment thereof is provided, wherein the method comprises culturing the host cell under conditions suitable for expression of the nucleic acid encoding the antibody, and isolating the antibody. In certain embodiments, the method further comprises recovering the anti-RAGE antibody from the host cell.

In one aspect, provided herein is a pharmaceutical composition comprising an anti-RAGE antibody or fragment thereof as described herein. In certain embodiments, the composition is suitable for subcutaneous administration. In certain embodiments, the composition is suitable for intravenous administration. In certain embodiments, the viscosity of the composition is less than about 10 cP at 25° C. In other embodiments, the viscosity of the composition is less than about 20 cP at 25° C., such as for example and not limitation, about 15 cP, about 16 cP, about 17 cP, about 18 cP, and about 19 cP. Any anti-RAGE antibodies known in the art or described herein may be formulated into the composition. In certain embodiments, the composition comprises a pharmaceutically acceptable carrier, such as for example and not limitation, a polymeric carrier. In certain embodiments, the composition may further comprise one or more of albumin, sucrose, trehalose, lactitol, gelatin, hydroxypropyl-β-cyclodextrin, methoxypolyethylene glycol and polyethylene glycol, arginine, and Tween.

In certain embodiments, the composition may further comprise an adjuvant.

In certain embodiments, the composition may further comprise an additional agent, such as for example and not limitation, a therapeutic agent, imaging agent, cytotoxic agent, angiogenesis inhibitors; kinase inhibitors; co-stimulation molecule blockers; adhesion molecule blockers; anti-cytokine antibody or functional fragment thereof; methotrexate; cyclosporin; rapamycin; FK506; detectable label or reporter; a TNF antagonist; an antirheumatic; a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist. Further examples are: latrepirdine, anti-Aβ-antibodies, beta-secretase inhibitors, tau-modulators, cognition enhancers like e.g. 5-HT6 antagonists, cholesterinase inhibitor (e.g., tactrine, donepezil, rivastigmine or galantamine), a partial NMDA receptor blocker (e.g., memantine), a glycosaminoglycan mimetic (e.g., tramiprosate), an inhibitor or allosteric modulator of gamma secretase (e.g., R-flurbiprofen), a luteinizing hormone blockade gonadotropin releasing hormone agonist (e.g., leuprorelin), a serotinin 5-HT1A receptor antagonist, a chelatin agent, a neuronal selective L-type calcium channel blocker, an immunomodulator, an amyloid fibrillogenesis inhibitor or amyloid protein deposition inhibitor (e.g., M266), another antibody (e.g., bapineuzumab), a 5-HT1a receptor antagonist, a PDE4 inhibitor, a histamine agonist, a receptor protein for advanced glycation end products, a PARP stimulator, a serotonin 6 receptor antagonist, a 5-HT4 receptor agonist, a human steroid, a glucose uptake stimulant which enhanced neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor (NGF) receptor agonist, and a gene therapy modulator.

In one aspect, provided herein is a subcutaneous administration device containing an anti-RAGE antibody or a composition comprising an anti-RAGE antibody, or fragment thereof, described herein. In certain embodiments, the device is for delivering to an individual a dose in the range of about 10 to about 1200 mg of the antibody per dose.

### B. Therapeutic and Diagnostic Methods

In one aspect, the disclosure concerns methods of inhibiting binding of RAGE to Erk/MAPK pathway proteins in a subject, said method comprising administering to the subject an effective amount of any of the anti-RAGE antibodies, or fragments thereof, described herein. In another aspect, the disclosure concerns methods of reducing at least one of chronic inflammation, immune suppression, cancer/tumor cell migration, cancer/tumor cell infiltration, suppressive immune cell infiltration into the tumor, tumor promoting immune cell infiltration, cancer/tumor growth, liver damage and fibrosis, neovascularization in the eye, respiratory diseases, infections, and/or cancer/tumor progression in a subject, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In one aspect, the disclosure concerns methods of inhibiting RAGE activity to prevent and/or treat cancer, such as for example and not limitation cancers characterized by high vascularization such as colorectal and renal cancers, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of inhibiting RAGE activity to alter a tumor microenvironment, such as for example and not limitation, to inhibit vascularization to a tumor, inhibit growth of the tumor, inhibit or decrease expression of growth factors within the tumor, decrease or alter myeloid lineage cell type infiltration and/or activity, decrease or alter mesenchymal/stromal lineage cell type infiltration and/or activity, and/or to promote T cell activity and infiltration into the tumor, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of inhibiting RAGE activity to prevent the development of chronic inflammation, said method comprising administering to the subject an effective amount of any of the anti-RAGE antibodies or fragments thereof described herein. In a related aspect, the disclosure concerns methods of inhibiting RAGE activity to maintain an acute inflammatory state, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of inhibiting RAGE activity to prevent liver damage and/or fibrosis, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of inhibiting RAGE activity to prevent neovascularization in the eye, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of inhibiting RAGE activity to prevent respiratory diseases and/or the development of respiratory diseases, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20). In some embodiments, the respiratory disease is allergic airway inflammation (AAI) and asthma, pulmonary fibrosis, lung cancer, chronic obstructive pulmonary disease (COPD), acute lung injury, pneumonia, cystic fibrosis, and bronchopulmonary dysplasia.

In another aspect, the disclosure concerns methods of inhibiting RAGE activity to prevent infections and/or the development of infections, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20). In some embodiments, the infectious disease is a result of infection with one or more of a virus, a bacteria, a fungus, a prion, a parasite, etc.

In one aspect, the disclosure concerns methods of treating a disease or disorder, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In one aspect, the disclosure concerns methods of treating cancer, such as for example and not limitation, cancers characterized by high vascularization such as colorectal and renal cancers, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In some embodiments, the cancer is a leukemia (e.g., acute lymphoblastic leukemia, acute myeloid leukemia, chronic lymphoblastic leukemia, chronic myeloid leukemia, hairy cell leukemia), adrenocortical carcinoma, anal cancer, an astrocytoma, Kaposi sarcoma, a lymphoma (e.g., AIDS-related lymphoma, primary CNS lymphoma, non-Hodgkin lymphoma, Hodgkin lymphoma, cutaneous T-cell lymphoma, mycosis fungoides), a skin cancer (e.g., basal cell carcinoma, melanoma, Merkel cell carcinoma), bile duct cancer, bladder cancer, bone cancer, a brain tumor, breast cancer, carcinoid tumor, carcinoma of unknown primary, a CNS cancer, cervical cancer, a chronic myeloproliferative neoplasm, colorectal cancer, craniopharyngioma, an embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma, gallbladder cancer, gastric cancer, gastrointestinal carcinoid tumor, gastrointestinal stromal tumor, germ cell tumor, head and neck cancer, hepatocellular carcinoma, Langerhans cell histiocytosis, hypopharyngeal cancer, islet cell tumor, renal cell cancer, laryngeal cancer, lip and oral cavity cancer, lung cancer (e.g., non-small cell lung cancer and small cell lung cancer), malignant mesothelioma, mouth cancer, multiple myeloma, a myelodysplastic syndrome, nasal cavity and paranasal sinus cancer, nasopharyngeal cancer, neuroblastoma, oral cancer, osteosarcoma, ovarian cancer, pancreatic cancer, papillomatosis, paraganglioma, parathyroid cancer, penile cancer, pharyngeal cancer, pheochromocytoma, pituitary tumor, primary peritoneal cancer, prostate cancer, rectal cancer, retinoblastoma, rhabdomyosarcoma, salivary gland cancer, sarcoma (e.g., rhabdomyosarcoma, vascular tumor, Ewing sarcoma, Kaposi sarcoma, osteosarcoma, uterine sarcoma, soft tissue sarcoma), Sezary syndrome, squamous cell carcinoma, testicular cancer, thymoma and thymic carcinoma, thyroid cancer, urethral cancer, uterine cancer, uterine sarcoma, vaginal cancer, vascular tumors, vulvar cancer, and Wilms tumor.

In some embodiments, the cancer is a colorectal cancer. There are four consensus molecular subtypes (CMS) with distinguishing features:
CMS1 (MSI Immune, 14%): hypermutated, microsatellite unstable, strong immune activation;
CMS2 (Canonical, 37%): epithelial, chromosomally unstable, marked WNT and MYC signaling activation;
CMS3 (Metabolic, 13%): epithelial, evident metabolic dysregulation; and
CMS4 (Mesenchymal, 23%): prominent transforming growth factor β activation, stromal invasion, and angiogenesis.
   Colorectal carcinoma samples with mixed features (13%) possibly represent a transition phenotype or intra-tumoral heterogeneity. In some embodiments, an anti-RAGE antibody disclosed herein is used to treat colorectal cancer in combination with a biologic immunotherapy customized for the CMS of the colorectal cancer.

In another aspect, the disclosure concerns methods of altering a tumor microenvironment, such as for example and not limitation, to inhibit vascularization to a tumor, inhibit growth of the tumor, inhibit or decrease expression of growth factors within the tumor, decrease or alter myeloid lineage cell type infiltration and/or activity, decrease or alter mesenchymal/stromal lineage cell type infiltration and/or activity, and/or to promote T cell activity and infiltration into the tumor, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of preventing the development of chronic inflammation, said method comprising administering to the subject an effective amount of any of the anti-RAGE antibodies or fragments thereof described herein. In a related aspect, the disclosure concerns maintaining an acute inflammatory state, said method comprising administering to the subject an effective amount of any of the anti-RAGE or fragments thereof antibodies described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of preventing liver damage and/or fibrosis, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of preventing neovascularization in the eye, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure concerns methods of preventing respiratory diseases and/or the development of respiratory diseases, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20). In some embodiments, the respiratory disease is allergic airway inflammation (AAI) and asthma, pulmonary fibrosis, lung cancer, chronic obstructive pulmonary disease (COPD), acute lung injury, pneumonia, cystic fibrosis, and bronchopulmonary dysplasia.

In another aspect, the disclosure concerns methods of preventing infections and/or the development of infections, said method comprising administering to the subject an effective amount of any of the anti-RAGE antibodies or fragments thereof described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20). In some embodiments, the infectious disease is a result of infection with one or more of a virus, a bacteria, a fungus, a prion, a parasite, etc.

In another aspect, the disclosure provides a method for reducing human RAGE activity comprising contacting human RAGE with any of the anti-RAGE antibodies or fragments thereof as described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure provides a method for decreasing human RAGE binding to at least one ligand selected from Erk, S100b, AGE, HMGB1 (amphoterin), S100A12 (EN-RAGE), S100A7 (psoriasin), S100P, S100A8/A9 complex calprotectin), amyloid-β-protein, Mac-1, phosphatidylserine, and S100A4. in a subject in need thereof, comprising the step of administering to the subject any of the anti-RAGE antibodies or fragments thereof as described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure provides a method of treating a subject for a disorder associated with RAGE activity comprising the step of administering alone, or in combination with other therapeutic agents, any of the anti-RAGE antibodies or fragments thereof as described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In another aspect, the disclosure provides a method for reducing RAGE activity in a subject suffering from a disorder in which RAGE activity is detrimental, comprising administering to the subject any of the anti-RAGE antibodies or fragments thereof as described herein, alone or in combination with other therapeutic agents. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In certain embodiments, the methods described herein further comprise administering to the subject an effective amount of a second medicament or therapeutic agent, wherein the anti-RAGE antibody or fragment thereof is the first medicament or therapeutic agent. In one embodiment, the second medicament or therapeutic agent is a cancer therapeutic. In certain embodiments, the subject or the individual is human. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In one aspect, the disclosure concerns a method of detecting RAGE protein in a sample suspected of containing the RAGE protein, the method comprising (a) contacting the sample with an anti-RAGE antibody or fragment thereof described herein; and (b) detecting formation of a complex between the anti-RAGE antibody or fragment thereof and the RAGE protein. In one embodiment, the anti-RAGE antibody or fragment thereof is detectably labeled.

The present disclosure also provides new diagnostic tests for characterizing an individual's risk of developing or having a disease or condition characterized by high levels of RAGE, as well as of identifying a subject with high levels of RAGE that responds, or is likely to respond, to certain treatments or therapeutics to lower RAGE levels. Still other diagnostic tests include monitoring levels of RAGE during treatment.

In certain embodiments, the methods described herein comprise identifying a subject that responds to anti-RAGE antibody treatment, comprising determining a level of RAGE in a sample from the subject using an antibody described herein and comparing the level of RAGE to a comparable sample obtained from the general population or a select population of human subjects. The level may also be compared to the level of RAGE in the subject before beginning anti-RAGE antibody treatment. Such comparison characterizes the subject's response to anti-RAGE antibody treatment.

In certain embodiments, the methods described herein comprise identifying a subject that responds to a treatment for a disease or condition characterized by high levels of RAGE (such as for example and not limitation, cancer, chronic inflammation, cancer/tumor cell migration, cancer/tumor cell infiltration, suppressive immune cell infiltration into the tumor, tumor promoting immune cell infiltration, cancer/tumor growth, liver damage and fibrosis, neovascularization in the eye, respiratory diseases, infections, cancer/tumor progression, and/or devascularized tumor microenvironment), comprising determining a level of RAGE in a sample from the subject using an antibody described herein and comparing the level of RAGE to a comparable sample obtained from the general population or a select population of human subjects. The level may also be compared to the level of RAGE in the subject before beginning anti-RAGE antibody treatment. Such comparison characterizes the subject's response to anti-RAGE antibody treatment.

The present disclosure also provides a method for monitoring over time the status of RAGE levels in a subject. The method comprises determining the level of RAGE in a sample taken from the subject at an initial time and in a corresponding sample taken from the subject at a subsequent time. An increase in the level of RAGE from the sample taken at the subsequent time as compared to the initial time indicates that the subject's risk of having or developing a future RAGE-related condition or disorder has increased. A decrease in the level of RAGE from the sample taken at the subsequent time as compared to the initial time indicates that that the subjects risk of having or developing a future RAGE-related condition or disorder has decreased.

In other embodiments, the present disclosure provides a method for evaluating therapy in a subject suspected of having or having a condition or disease associated with high levels of RAGE. The method comprises determining the level of RAGE in a sample taken from the subject prior to therapy and a corresponding sample taken from the subject during or following therapy. A decrease in the level of RAGE in the sample taken after or during therapy as compared to the level of RAGE in the sample taken before therapy is indicative of a positive effect of the therapy on the RAGE-related disease or condition in the treated subject.

In one aspect, the present disclosure provides a method for diagnosing a condition or disease associated with high levels of RAGE in a subject, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) having a condition or disease associated with high levels of RAGE when the RAGE level in the subject is greater than or equal to the RAGE level in the comparable sample or (ii) not having a condition or disease associated with high levels of RAGE when the RAGE level in the subject is less than the RAGE level in the comparable sample.

In another aspect, the present disclosure provides a method for detection of a condition or disease associated with high levels of RAGE in a subject in a subject, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) having a condition or disease associated with high levels of RAGE when the RAGE level in the subject is greater than or equal to the RAGE level in the comparable sample or (ii) not having a condition or disease associated with high levels of RAGE when the RAGE level in the subject is less than the RAGE level in the comparable sample.

In one aspect, the present disclosure provides a method for classifying a subject as having a condition or disease associated with high levels of RAGE or not having such disease or condition, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) having a condition or disease associated with high levels of RAGE when the RAGE level in the subject is greater than or equal to the RAGE level in the comparable sample or (ii) not having a condition or disease associated with high levels of RAGE when the RAGE level in the subject is less than the RAGE level in the comparable sample.

In another aspect, the present disclosure provides a method for monitoring a condition or disease associated with high levels of RAGE in a subject, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) having or continuing to have a condition or disease associated with high levels of RAGE when the RAGE level in the subject is greater than or equal to the RAGE level in the comparable sample or (ii) not having or no longer having a condition or disease associated with high levels of RAGE when the RAGE level in the subject is less than the RAGE level in the comparable sample.

In one aspect, the present disclosure provides a method for selecting a subject for a clinical trial for anti-RAGE antibody therapeutic compositions and/or methods, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) being suitable for the clinical trial when the RAGE level in the subject is greater than or equal to the RAGE level in the comparable sample or (ii) not being suitable for the clinical trial when the RAGE level in the subject is less than the RAGE level in the comparable sample.

In one aspect, the present disclosure provides a method for treating and/or preventing a condition or disease associated with high levels of RAGE in a subject, comprising the steps of:
a) measuring the level of RAGE in a sample collected from the subject;
b) comparing the RAGE level obtained from the subject to a RAGE level in a comparable sample obtained from the general population or a select population of human subjects;
c) identifying the subject as (i) being suitable for treatment of, or having a condition or disease associated with high levels of RAGE when the RAGE level in the subject is greater than or equal to the RAGE level in the comparable sample or (ii) not being suitable for treatment of, or not having a condition or disease associated with high levels of RAGE when the RAGE level in the subject is less than the RAGE level in the comparable sample; and
d) utilizing appropriate therapeutic and/or prophylactic compositions and/or methods if the subject has a condition or disease associated with high levels of RAGE or is suitable for treatment.

In another aspect, the disclosure provides a method for predicting the risk of developing a condition or disease associated with high levels of RAGE in a subject, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) being at risk for having or developing a condition or disease associated with high levels of RAGE when the RAGE level in the subject is greater than or equal to the RAGE level in the comparable sample or (ii) not being at risk for having or developing a condition or disease associated with high levels of RAGE when the RAGE level in the subject is less than the RAGE level in the comparable sample.

In one aspect, the disclosure provides a method for predicting a subject's response to a treatment for a condition or disease associated with high levels of RAGE in a subject, comprising the steps of:
a) measuring the level of RAGE in a sample collected from the subject;
b) comparing the RAGE level obtained from the subject to a RAGE level in a comparable sample obtained from the general population or a select population of human subjects;
c) identifying the subject as (i) being at risk for having or developing a condition or disease associated with high levels of RAGE when the RAGE level in the subject is greater than or equal to the RAGE level in the comparable sample or (ii) not being at risk for having or developing a condition or disease associated with high levels of RAGE when the RAGE level in the subject is less than the RAGE level in the comparable sample; and
(d) predicting the subject's response to the treatment for severe or lethal GVHD based on the identification step.

In one aspect, the disclosure provides a method for monitoring the potential to develop a condition or disease associated with high levels of RAGE in a subject, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) having or being at risk for having or developing a condition or disease associated with high levels of RAGE when the RAGE level in the subject is greater than or equal to the RAGE level in the comparable sample or (ii) not having or not being at risk for having or developing a condition or disease associated with high levels of RAGE when the RAGE level in the subject is less than the RAGE level in the comparable sample.

In another aspect, the disclosure provides a method for monitoring the success and/or efficacy of treatment and/or prophylaxis of a subject who has or who is likely to develop a condition or disease associated with high levels of RAGE, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the treatment and/or prophylaxis as (i) being successful when the RAGE level in the subject is greater than or equal to the RAGE level in the comparable sample or (ii) not being successful when the RAGE level in the subject is less than the RAGE level in the comparable sample. Optionally, steps a, b, and/or c may be repeated during and after the course of the treatment and/or prophylaxis.

In one aspect, the disclosure provides a method for selecting a subject for a treatment and/or prophylaxis of a condition or disease associated with high levels of RAGE, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) being suitable for the treatment and/or prophylaxis when the RAGE level in the subject is greater than or equal to the RAGE level in the comparable sample or (ii) not being suitable for the treatment and/or prophylaxis when the RAGE level in the subject is less than the RAGE level in the comparable sample. Optionally, steps a, b, and/or c may be repeated during and after the course of the treatment and/or prophylaxis.

In a related aspect, the disclosure provides a method for prognosis of a condition or disease associated with high levels of RAGE, comprising the steps of:
a) measuring the level of RAGE in a sample collected from the subject;
b) comparing the RAGE level obtained from the subject to a RAGE level in a comparable sample obtained from the general population or a select population of human subjects; and
c) identifying the subject as (i) likely to develop a condition or disease associated with high levels of RAGE when the RAGE level in the subject is greater than or equal to the RAGE level in the comparable sample or (ii) not likely to develop a condition or disease associated with high levels of RAGE when the RAGE level in the subject is less than the RAGE level in the comparable sample.

In any of the above embodiments, steps (b) and/or (c) may be performed by a computer.

In any of the foregoing methods, step (a) further comprises obtaining a blood sample from the subject and subsequently isolating RAGE. In any of the foregoing methods, step (b) further comprises obtaining a blood sample from a member of the general population or select population and subsequently isolating RAGE.

In any of the foregoing methods, steps (a) and/or (b) further comprise determining the level of RAGE in the sample.

In any of the foregoing methods, determination of the level of RAGE further comprises isolating nucleic acid and/or protein from the sample.

In any of the foregoing methods, the method further comprises treatment and/or prophylaxis of subjects determined to have, or be at risk for, a condition or disease associated with high levels of RAGE.

In one embodiment of any of the foregoing methods, the treatment is prophylactic and comprises administration of appropriate prophylactically effective pharmaceutical compositions and/or use of appropriate prophylactically effective methods. In one embodiment, the prophylactic treatment comprises immunosuppressive drugs, anti-inflammatory drugs, cytototoxic drugs, immunomodulatory drugs, etc. In other embodiments, the prophylactic treatment comprises at least one anti-RAGE antibody or fragment thereof as described herein. In still other embodiments, the prophylactic treatment comprises a therapeutic agent and at least one anti-RAGE antibody or fragment thereof as described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

In one embodiment of any of the foregoing methods, the treatment comprises administration of appropriate therapeutically effective pharmaceutical compositions and/or use of appropriate therapeutically effective methods. In one embodiment, the therapeutically effective compositions comprise cancer therapeutics and/or immune modulating therapies (e.g., for example and not limitation, checkpoint inhibitors such as nivolumab and pembrolizumab, anti-cancer agents which induce immunogenic cell death (ICD) such as platinum compound, taxanes, and alkylating agents (e.g., oxaliplatin, paclitaxel, and cyclophosphamide, respectively), VEGF pathway and vascular targeted antibodies and tyrosine kinase inhibitors (TKI) such as bevacizumab and sunitinib, respectively, antimetabolites such as 5-fluorouracil and gemcitabine, antibody therapies that engage an ADCC response such as cetuximab and trastuzumab). In another embodiment, the therapeutically effective methods comprise use of high doses of steroids (e.g., corticosteroids) and/or use of immunosuppressive therapies and/or use of cancer therapeutics. In other embodiments, the therapeutic treatment comprises at least one anti-RAGE antibody or fragment thereof as described herein. In still other embodiments, the therapeutic treatment comprises a therapeutic agent and at least one anti-RAGE antibody or fragment thereof as described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

The treatment and/or prophylaxis of a subject having or at risk for having a condition and/or disease associated with high levels of RAGE may be administered to the subject via any suitable route of administration. The effective amount or dose of such treatment and/or prophylaxis administered should be sufficient to provide a therapeutic or prophylactic response in the subject over a reasonable time frame. For example, the dose of immunosuppressive drug should be sufficient to decrease symptoms of such condition and/or disease along with decreasing the level of RAGE. The dose will be determined by the efficacy of the particular active agent and the condition of the subject (e.g., human), as well as the body weight of the subject (e.g., human) to be treated.

Any embodiment described herein, or any combination thereof, applies to any and all anti-RAGE antibodies, methods and uses of the disclosure described herein. In some embodiments, the anti-RAGE antibodies or fragments thereof comprise at least one of the amino acid sequences of SEQ ID NOs: 2-17 and/or amino acid sequences having at least 90%, 91%, 92%, 93%, 94%, 95%, 96%, 97%, 98%, 99%, or 100% sequence identity to the amino acid sequences of SEQ ID NOs: 2-17. In some embodiments, the anti-RAGE antibodies or fragments thereof bind to at least one epitope of the human RAGE protein (e.g., SEQ ID NO: 1), such as for example and not limitation, an epitope present in the V and/or C1 domain of the human RAGE protein (e.g., SEQ ID NOs: 19 and/or 20).

It is also intended that the isolated antibodies or fragments thereof that interact with RAGE of the present application may be a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the disclosure may include, but are not limited to, glucose, galactose, mannose, fucose, n-acetylglucosamine, and sialic acid.

The antibodies or fragments thereof of the present application comprise a heavy chain constant region, such as an IgG1, IgG2, IgG3, IgG4, IgA, IgE, IgM, or IgD constant region. Furthermore, the antibody can comprise a light chain constant region, either a kappa light chain constant region or a lambda light chain constant region. Particularly, the antibody comprises a kappa light chain constant region. Alternatively, the antibody portion can be, for example, a Fab fragment or a single chain Fv fragment. Replacements of amino acid residues in the Fc portion to alter antibody effector function are known in the art (U.S. Pat. Nos. 5,648,260; 5,624,821). The Fc portion of an antibody mediates several important effector functions. e.g., cytokine induction, ADCC, phagocytosis, complement dependent cytotoxicity (CDC), and half-life/clearance rate of antibody and antigen-antibody complexes. In some cases these effector functions are desirable for therapeutic antibodies but in other cases might be unnecessary or even deleterious, depending on the therapeutic objectives. Certain human IgG isotypes, particularly IgG1 and IgG3, mediate ADCC and CDC via binding to Fcγ receptors and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

It is contemplated that when used to treat various diseases, the compositions and methods of the present disclosure can be combined with other therapeutic agents suitable for the same or similar diseases. Also, two or more embodiments of the disclosure may be also co-administered to generate additive or synergistic effects. When co-administered with a second therapeutic agent, the embodiment of the disclosure and the second therapeutic agent may be simultaneously or sequentially (in any order). Suitable therapeutically effective dosages for each agent may be lowered due to the additive action or synergy.

As a non-limiting example, the disclosure can be combined with other therapies that block inflammation (e.g., via blockage of IL1, INFα/β, IL6, TNF, IL13, IL23, etc.).

The compositions and methods of the disclosure can be also administered in combination with an anti-tumor antibody or an antibody directed at a pathogenic antigen or allergen.

The compositions and methods of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments of the disclosure can be also combined with other treatments that possess the ability to modulate natural killer T-cell function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents.

Therapeutic methods of the disclosure can be combined with additional immunotherapies and therapies. For example, when used for treating cancer, the anti-RAGE antibodies or fragments thereof of the disclosure can be used in combination with conventional cancer therapies, such as, e.g., surgery, radiotherapy, chemotherapy, or combinations thereof, depending on type of the tumor, patient condition, other health issues, and a variety of factors. In certain aspects, other therapeutic agents useful for combination cancer therapy with the inhibitors of the disclosure include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, and placental proliferin-related protein. In one embodiment, the anti-RAGE antibodies or fragments thereof of the disclosure can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases, and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

The compositions of the disclosure can comprise a carrier and/or excipient. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Those of relevant skill in the art are well able to prepare suitable solutions.

In one embodiment of any of the compositions of the disclosure, the composition is formulated for delivery by a route such as, e.g., oral, topical, rectal, mucosal, sublingual, nasal, naso/oro-gastric gavage, parenteral, intraperitoneal, intradermal, transdermal, intrathecal, nasal, and intracheal administration. In one embodiment of any of the compositions of the disclosure, the composition is in a form of a liquid, foam, cream, spray, powder, or gel. In one embodiment of any of the compositions of the disclosure, the composition comprises a buffering agent (e.g., sodium bicarbonate).

Administration of the compounds and compositions in the methods of the disclosure can be accomplished by any method known in the art. Non-limiting examples of useful routes of delivery include oral, rectal, colonic (by enema), and via naso/oro-gastric gavage, as well as parenteral, intraperitoneal, intradermal, intravenous, subcutaneous, transdermal, intrathecal, nasal, and intratracheal administration. The active agent may be systemic after administration or may be localized by the use of regional administration, intramural administration, or use of an implant that acts to retain the active dose at the site of implantation. The carrier material should be non-toxic to the subject/patient.

The useful dosages of the compounds and formulations of the disclosure will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve a therapeutic effect. While it is possible to use a compound of the present disclosure for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerin, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethyl sulfoxide (DMSO), using surfactants, such as TWEEN® 80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Parenteral administration, generally characterized by injection, either subcutaneously, intramuscularly or intravenously, is often contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol, or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate, and cyclodextrins.

Implantation of a slow-release or sustained-release system, such that a constant level of dosage is maintained (e.g., U.S. Pat. No. 3,710,795) is also contemplated herein. Briefly, an anti-RAGE antibody disclosed herein is dispersed in a solid inner matrix (e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate) that is surrounded by an outer polymeric membrane (e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer) that is insoluble in body fluids. The agent diffuses through the outer polymeric membrane in a release rate controlling step. The percentage of active agent contained in such parenteral compositions is highly dependent on the specific nature thereof, as well as the activity of the agent and the needs of the subject.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial may contain, by way of example and without limitation, a single dosage (10-1000 mg, such as 10-500 mg) or multiple dosages of the agent. The lyophilized powder can be stored under appropriate conditions, such as at about 4° C. to room temperature. Reconstitution of this lyophilized powder with water for injection provides a formulation for use in parenteral administration. The precise amount depends upon the selected agent. Such amount can be empirically determined.

The inventive composition or pharmaceutically acceptable derivatives thereof may be formulated as aerosols for application e.g., by inhalation or intranasally. These formulations can be in the form of an aerosol or solution for a nebulizer, or as a microfine powder for insufflation, alone or in combination with an inert carrier such as lactose. In such a case, the particles of the formulation will, by way of example and without limitation, have diameters of less than about 50 microns, such as less than about 10 microns.

### Kits

In one aspect, the present disclosure provides a kit for diagnosing and/or detecting a condition or disease associated with high levels of RAGE in a subject, said kit comprising antibodies directed towards RAGE, wherein the antibodies can be used to determine the expression level of RAGE. The kit can also comprise a detection means. The kit may further optionally include control antibodies.

In some embodiments, these kits comprise detection reagents that specifically bind RAGE (e.g., SEQ ID NO: 1) and fragments thereof. The kits typically include an antibody that specifically binds to polypeptides of the disclosure, such as for example and not limitation, RAGE and fragments thereof and a label for detecting the presence of the probe. The kits may include several antibodies specific for the polypeptides of the disclosure. The kits may further comprise control antibody in order to provide a control level of the protein, and/or other standards or controls. The antibodies are optionally detectably labeled.

The kit may contain in separate containers a antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions for carrying out the assay may also be included in the kit. The assay may, for example and not limitation, be in the form of a sandwich ELISA or protein antibody array.

Reagents for detecting biomarkers of the present disclosure can be immobilized on a solid matrix such as a porous strip to form at least one biomarker detection site. The measurement or detection region of the porous strip may include a plurality of sites containing an antibody. A test strip may also contain sites for negative and/or positive controls. Alternatively, control sites can be located on a separate strip from the test strip. Optionally, the different detection sites may contain different amounts of immobilized antibodies, e.g., a higher amount in the first detection site and lesser amounts in subsequent sites. Upon the addition of test sample, the number of sites displaying a detectable signal provides a quantitative indication of the amount of biomarker present in the sample. The detection sites may be configured in any suitably detectable shape and are typically in the shape of a bar or dot spanning the width of a test strip.

A kit can further include protein isolation or purification means as well as positive and negative controls. A kit can also include a notice associated therewith in a form prescribed by a governmental agency regulating the manufacture, use or sale of diagnostic kits. Detailed instructions for use, storage and trouble-shooting may also be provided with the kit. A kit can also be optionally provided in a suitable housing that is useful for robotic handling in a high throughput setting.

The components of the kit may be provided as dried powder(s). When reagents and/or components are provided as a dry powder, the powder can be reconstituted by the addition of a suitable solvent. It is envisioned that the solvent may also be provided in another container. The container will generally include at least one vial, test tube, flask, bottle, syringe, and/or other container means, into which the solvent is placed, optionally aliquoted. The kits may also comprise a second container means for containing a sterile, pharmaceutically acceptable buffer and/or other solvent.

Where there is more than one component in the kit, the kit also will generally contain a second, third, or other additional container into which the additional components may be separately placed. However, various combinations of components may be comprised in a container.

Such kits may also include components that preserve or maintain proteins, such as reagents that protect against protein degradation. Any of the compositions or reagents described herein may be components in a kit.

In some embodiments, the kit further comprises an apparatus for collecting a blood sample from a subject. In other embodiments, the kit further comprises instructions for using the collection apparatus and/or the reagents comprising the kit.

### EXAMPLES

### Example 1. Generation of Anti-RAGE Antibodies

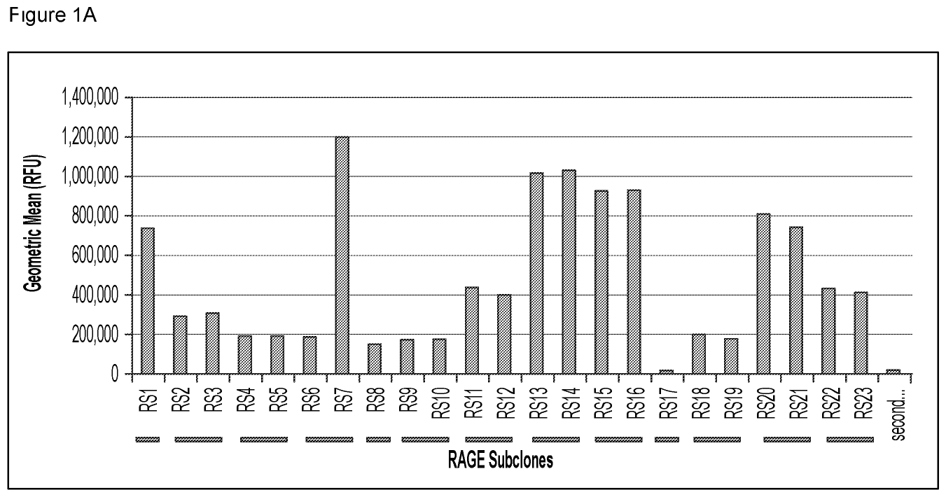

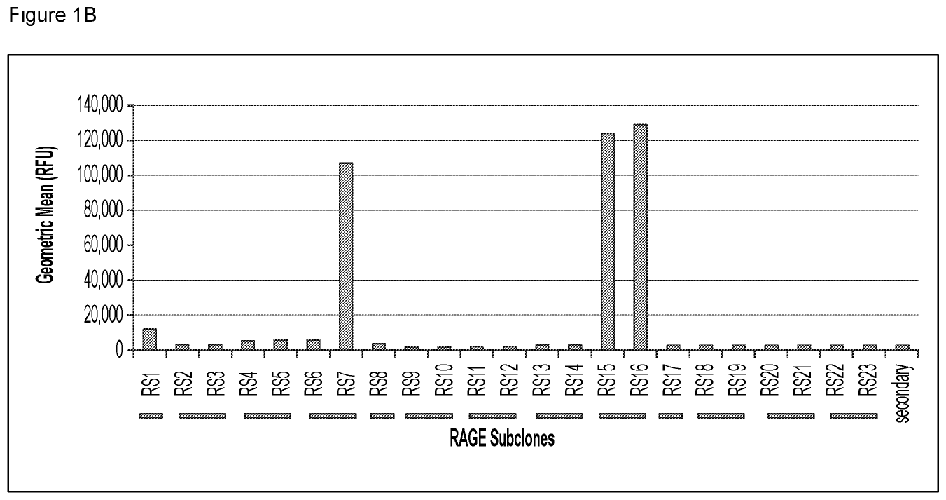

Mouse antibodies were generated against the purified extracellular domain of human RAGE using a 28 day RIMMS (Rapid Immunization Multiple Sites) protocol followed by a single boost in BALB/c mice. The hybridoma was generated from the fusion of splenocytes and lymphocytes from the immunized mouse with mouse myeloma cell line NS1. Hybridoma clones were assessed first by ELISA with hybridoma clone supernatants against purified human RAGE extracellular domain (ECD). ELISA positive clone supernatants were then tested by flow cytometry for IgG binding to human RAGE-transfected cells (FIG. 1A). ELISA and flow positive clones were subcloned and then evaluated for IgG expression, RAGE binding by ELISA and flow cytometry, and RAGE inhibitory activity in vitro. The cross reactivity of the mouse antibodies against human RAGE was assessed by flow cytometry binding of subclones to cells transfected with mouse RAGE (FIG. 1B). Three subclones were identified as binding to mouse RAGE and being cross-reactive with human RAGE:

### Example 2. Determination of Antibody Binding to RAGE Protein Domains

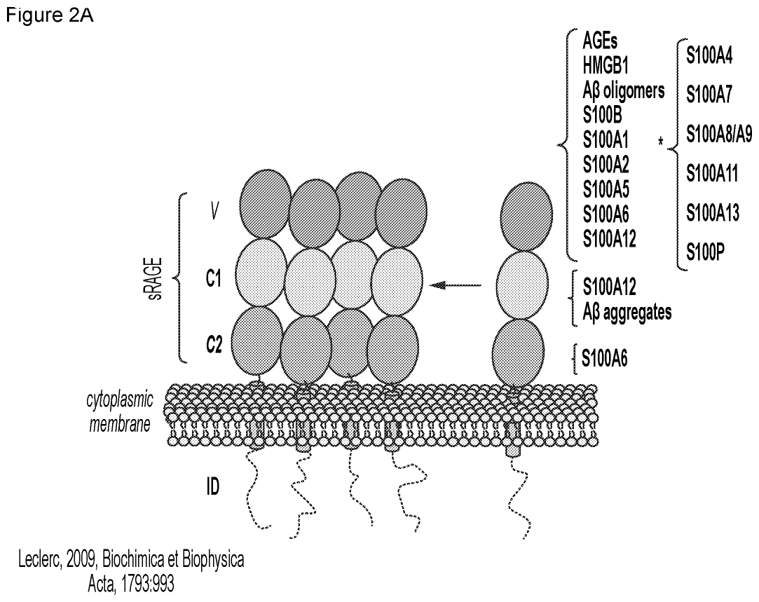

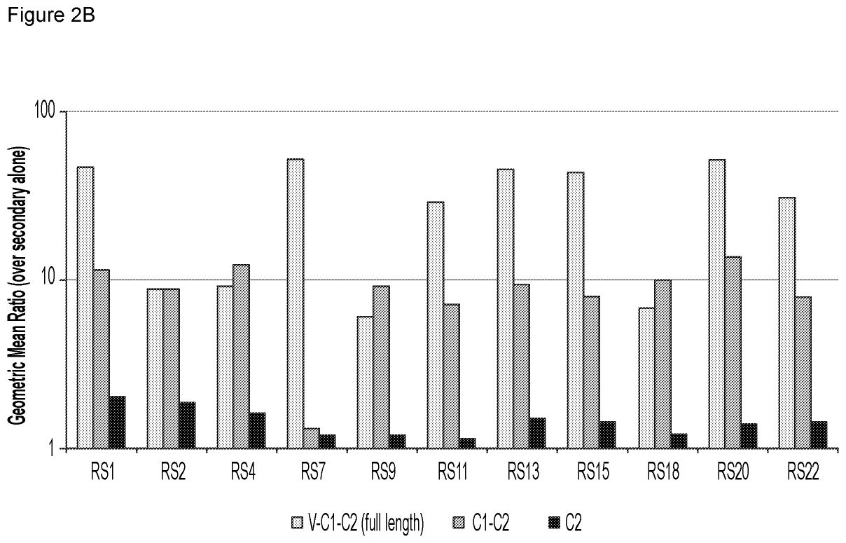

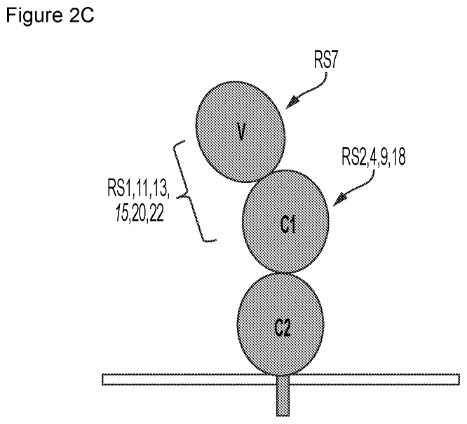

The extracellular portion of the RAGE protein contains three domains with similarities to the IgG family. The three domains are V, C1, and C2 (variable, constant 1, and constant 2, respectively). RS07 binds the V domain while RS15 binds an integrated V-C1 domain (FIG. 2B). RAGE constructs used in the present exemplary embodiment include a V-C1-C2 construct starting at amino acid 23 of SEQ ID NO:1 (AQNITA . . . ), immediately following the native signal sequence. The C1-C2 construct starts at amino acid 117 of SEQ ID NO:1 (VYQIPG . . . ), and the C2 construct starts at amino acid 249 of SEQ ID NO:1 (VAPGG . . . ). Results of the mapping experiment are shown in FIG. 2B and summarized in FIG. 2C. Domains were predicted on Genbank and by crystal structure. To map antibody binding to individual RAGE domains, the IL2 signal sequence was cloned into pEGFP-N2, into which portions of RAGE were cloned in-frame with IL2 signal sequence (IL2ss) at the 5′ end and in-frame with eGFP at the 3′ end. In addition to the extracellular portions of RAGE, all constructs contain the RAGE transmembrane and C-terminal regions, so the GFP signal should be intracellular, while the IL2ss ensures the RAGE V, C1, and/or C2 motifs are extracellular, and accessible to antibodies. The ABC construct in FIG. 2B includes the V, C1, and C2 domains, and initiates at aa 23 of SEQ ID NO: 1. The BC construct in FIG. 2B includes the C1 and C2 domains, and initiates at aa 116 of SEQ ID NO: 1. The C construct of FIG. 2B includes the C2 domain, and initiates at aa 246 of SEQ ID NO: 1.

### Example 3. In Vitro Activity of Antibodies

### Migration

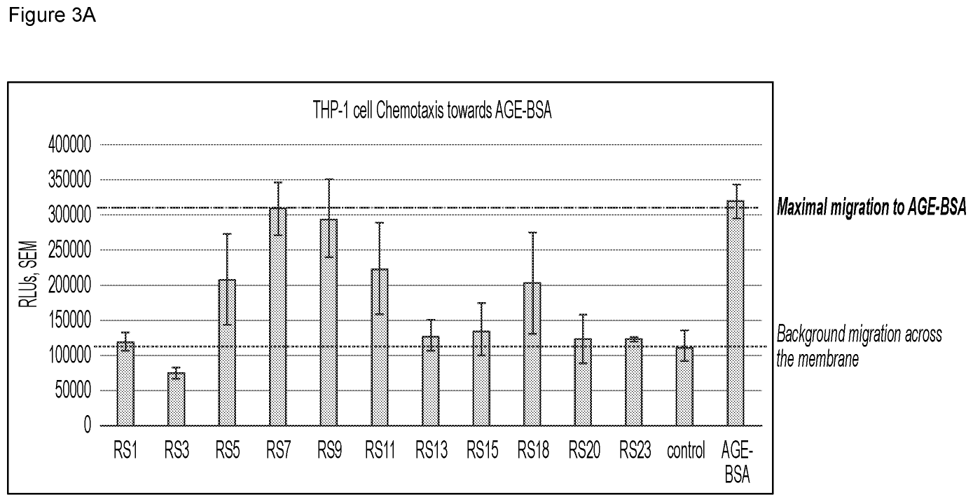

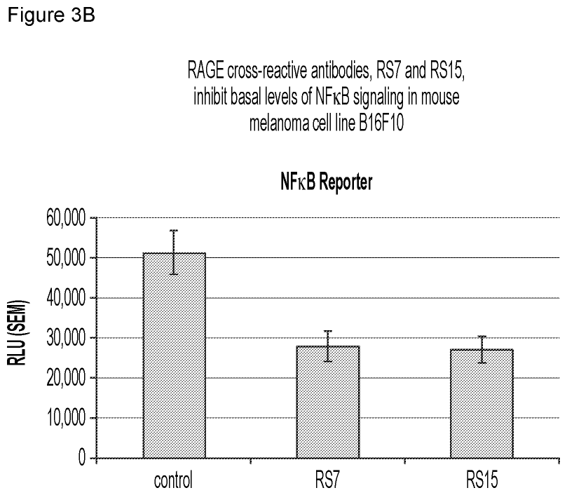

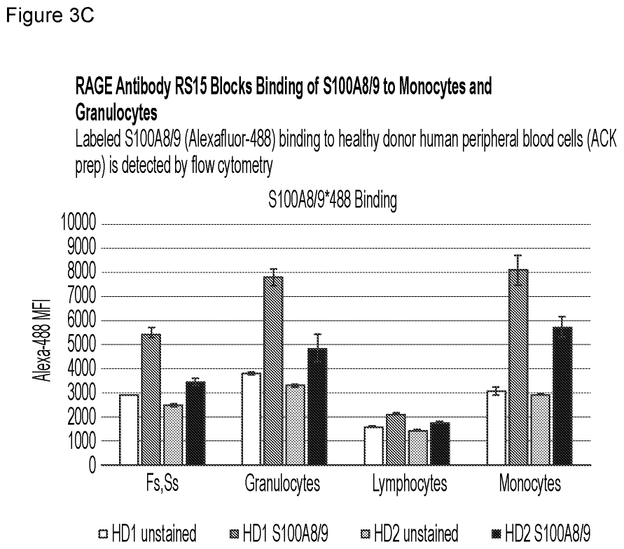

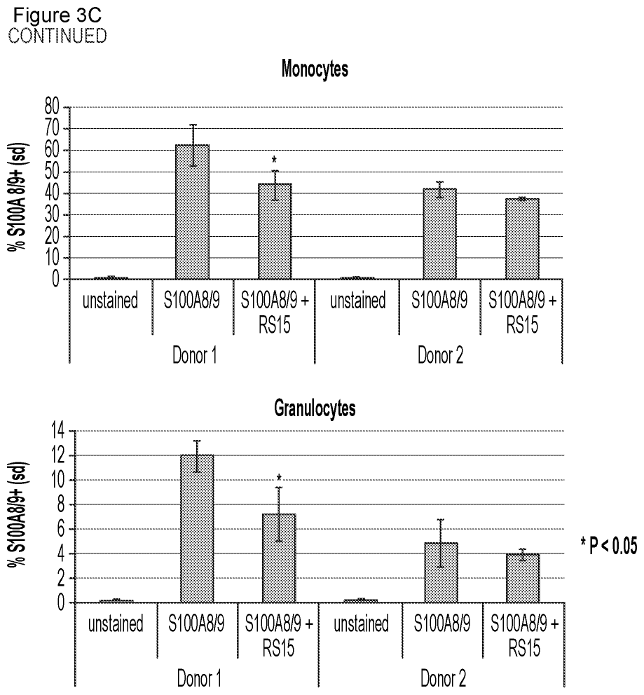

The ability of RAGE antibody clones to inhibit cell migration or chemotaxis was tested in human cell line THP-1. Some clones, including RS15, but not RS07, inhibited the ability of THP-1 cells to migrate towards RAGE ligand AGE-BSA (glycolated BSA, FIG. 3A). FIG. 3B shows that RS7 and RS15 inhibit basal levels of NFkB signaling in mouse melanoma line B16F10. FIG. 3C shows that RAGE antibody RS15 can block binding of S100A8/9 to monocytes and granulocytes. Here, labeled S100A8/9 (Alexafluor-488) binding to healthy donor human peripheral blood cells (ACK prep) was detected by flow cytometry.

### Erk Signaling

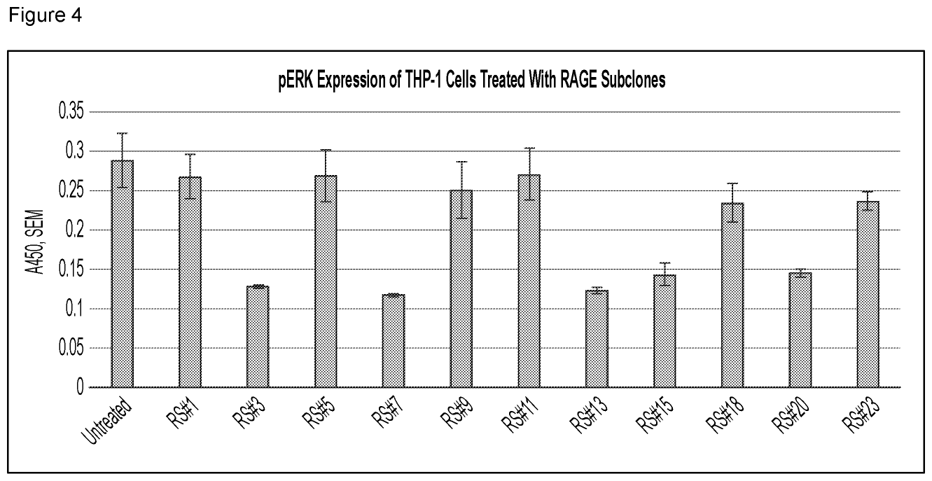

The ability of RAGE antibody clones to inhibit basal Erk activation levels was evaluated (phosphorylated Erk) in human THP-1 cells. Some clones, including both RS07 and RS15, inhibited activated Erk levels (FIG. 4).

### RAGE Human Tumor Expression

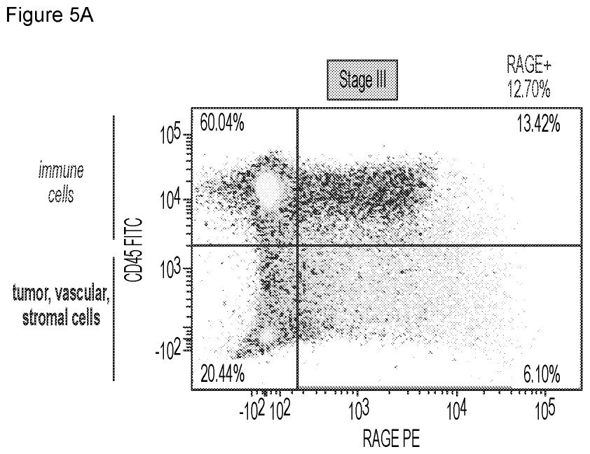

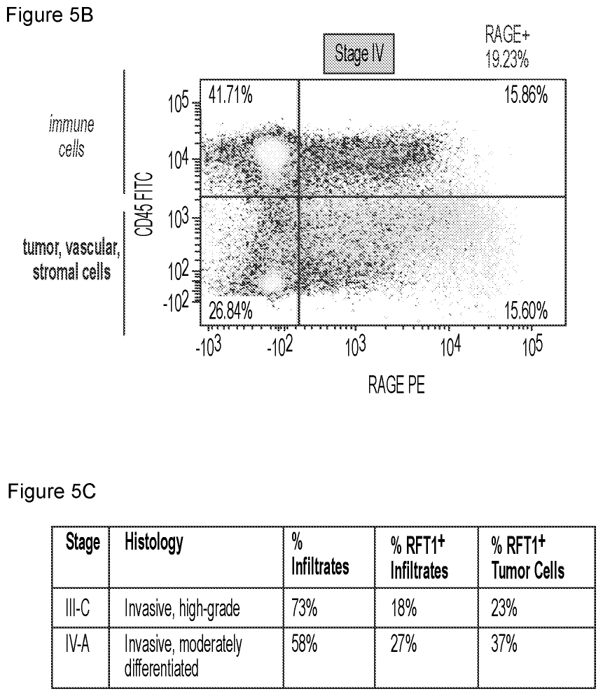

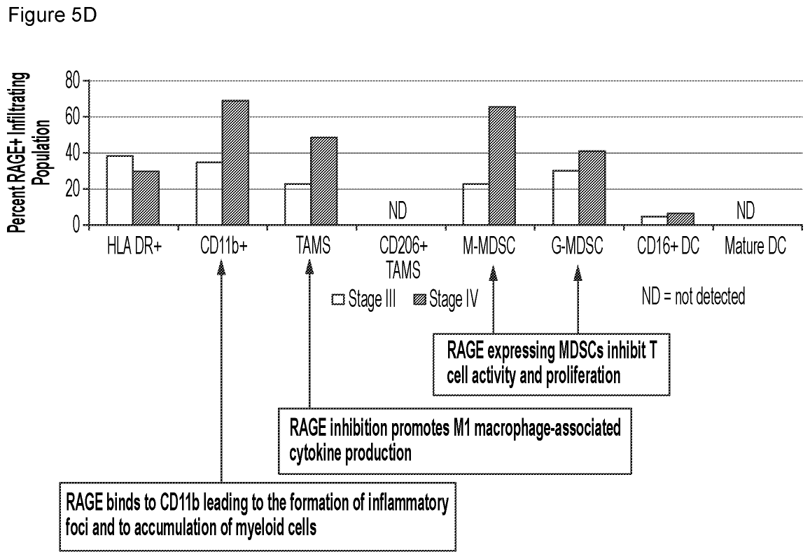

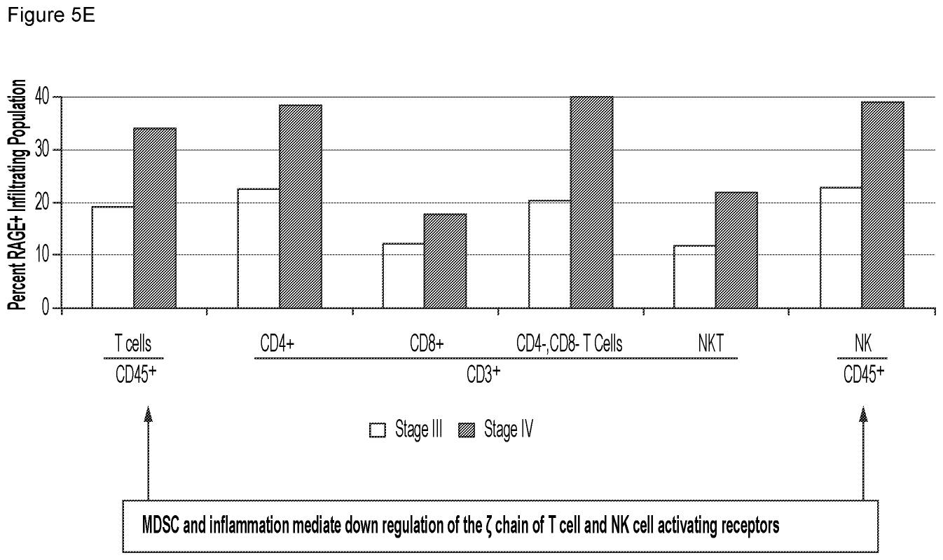

Using flow cytometry, RAGE expression was detected in two human colorectal cancer samples using RS15, also referred to as RFT01 (FIG. 5A). Analysis of specific cell types demonstrated that RAGE is expressed on multiple myeloid and lymphoid lineages infiltrating the tumor samples (FIGS. 5B and 5C, respectively). FIG. 5D shows the effects of anti-RAGE binding in colorectal cancer myeloid lineage cells in Stage III and Stage IV cancers. As can be seen in FIG. 5D, RAGE-expressing MDSCs inhibit T cell activity and proliferation, RAGE inhibition promotes M1 macrophage-associated cytokine production, and RAGE binds to CD11b leading to the formation of inflammatory foci and to accumulation of myeloid cells. The cells were identified by the markers in Table 1 below:

TABLE 1

| Myeloid | |
|---|---|
| Cell Type | Markers |
| M-MSDC | CD45+ CD11b+ CD14+ CD15+/− HLA-DRlow |
| G-MSDC | CD45+ CD11b+ CD14− CD15+ HLA-DR− |
| CD16+ DC | CD45+ CD11b+/− CD14− CD15− HLA-DR+ CD16+ |
| Mature DC | CD45+ HLA-DR+ CD83+ CD206+ |
| TAMs | CD45+ CD68+ HLA-DR+ |
| CD206+ TAMs | CD45+ CD68+ HLA-DR+ CD206+ |

FIG. 5E shows the effects of anti-RAGE binding in colorectal cancer lymphoid cells for Stage III and Stage IV cancers. Specifically, higher percentages of RAGE+ infiltrating cells were found in all cell types in Stage IV cancers. And, MDSC and inflammation were found to mediate down regulation of the z chain of T cell and NK cell activating receptors. The cancers were identified by the markers in Table 2 below:

TABLE 2

| Lymphoid Cell Type | Markers |
|---|---|
| T cells | CD45+ CD3+ |
| CD4+ T cell | CD45+ CD3+ CD4+ |
| CD8+ T cell | CD45+ CD3+ CD8+ |
| CD4−, CD8− T cells | CD45+ CD3+ CD4− CD8− |
| NKT | CD45+ CD3+ CD56+ |
| NK | CD45+ CD3− CD56+ |

### Example 4. In Vivo Activity of Antibodies

### RFT01 Treatment—Composition of Tumor Infiltrating Immune Cells

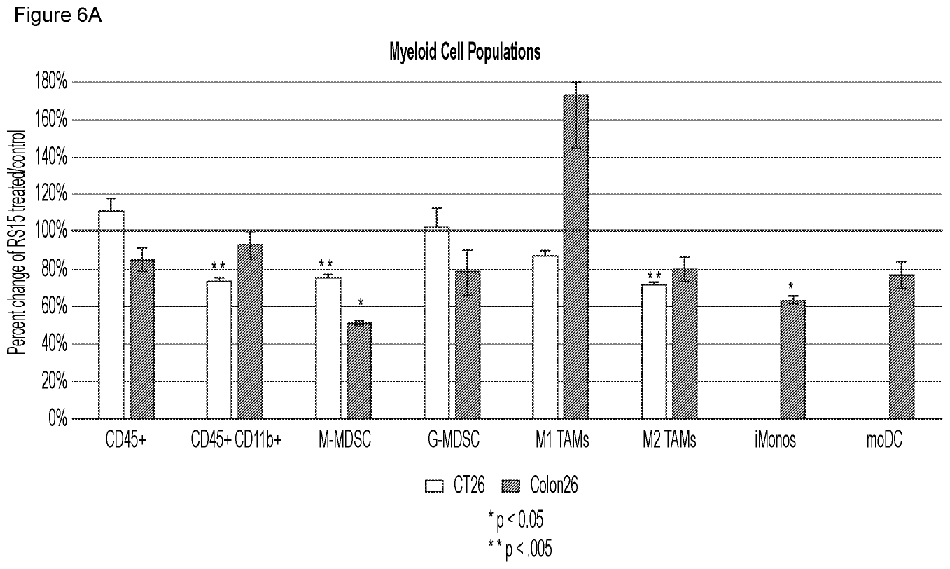

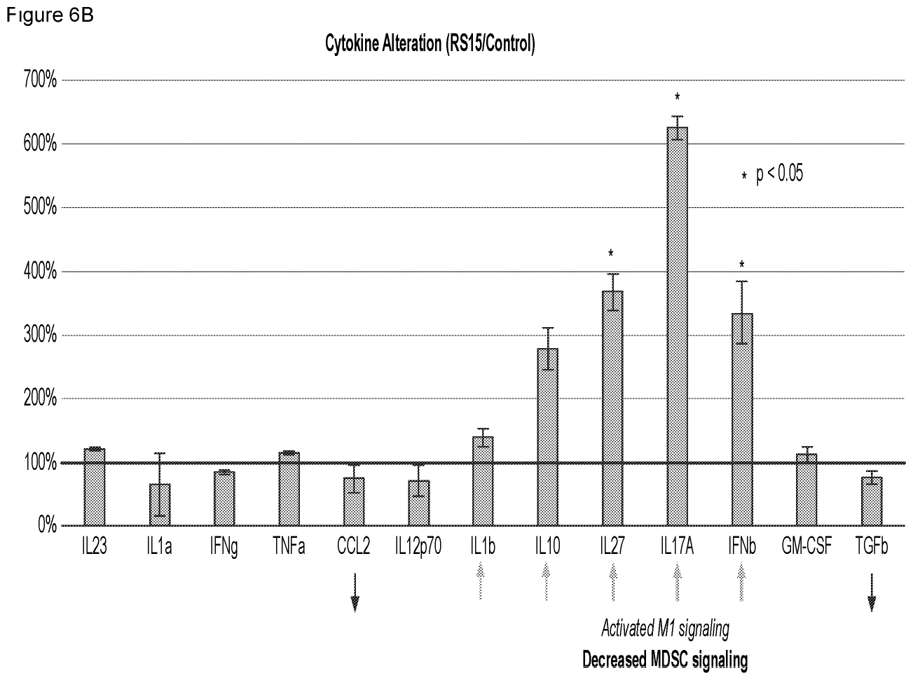

The effect of RFT01 treatment in vivo was assessed in two mouse syngeneic models of colon cancer, CT26 and Colon-26. RS15 treatment of CT26 tumor-bearing mice and Colon-26 (colon-26 adenocarcinoma) tumors resulted in a decrease in potentially immune inhibitory myeloid population infiltrating the tumor (FIG. 6A), specifically a decrease in total myeloid cells, M-MSDC, and M2_TAMs in CT26 tumor bearing mice, and a decrease in M-MSDC, iMonos, and an increase in M1 TAMs in Colon-26 mice and Colon-26 tumors. Further, RS15 treatment was shown to have effects on myeloid-associated cytokines in Colon-26 tumors (FIG. 6B). Specifically, IL27, IL17A, IFNβ, IL1β, and IL10 were increased, while TGFβ and CCL2 were decreased. IL27 is predominantly expressed by APCs, including macrophage and dendritic cells, and shows antitumor activity through direct and indirect mechanisms involving stimulation of NK cells and inhibition of angiogenesis. IL17A promotes CD8+ T cell cytotoxicity, and IL17 levels are increased in RAGE knockout mice. IFNβ is known to promote anti-inflammatory conditions. IL1p is a marker for M1 activated macrophages. IL10 is stimulated by IL27 and is known to be anti-inflammatory, though it can be immune stimulatory or inhibitory depending on the antigenic environment. TGFβ promotes a protumor microenvironment and is associated with CMS4 type colorectal cancer. Finally, CCL2 is expressed by monocytes, macrophage, and dendritic cells and is associated with cachexia, recruitment of macrophage and other immune cells to the tumor, and also contributes to tumor vascularization and growth.

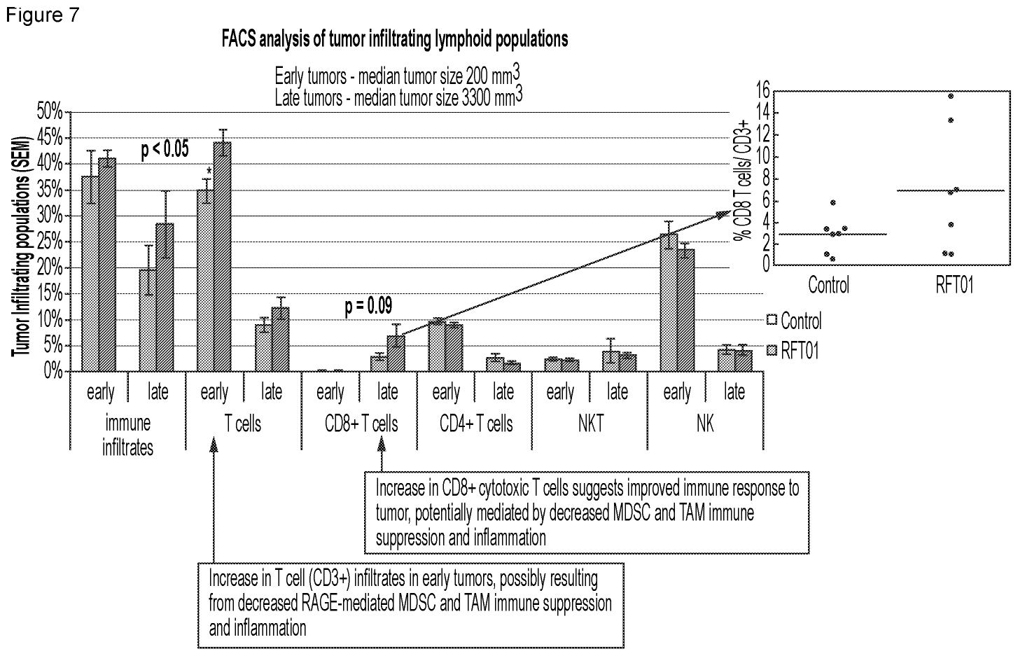

RFT01 treatment also increased the number of T cells infiltrating the tumor (FIG. 7), potentially indicating an immune response against the tumor. In early stage tumors (median tumor size 200 mm³), the number of CD3+ T cells increased and in late stage tumors (median tumor size 3300 mm³), the number of CD8+ T cells increased. The increase in CD3+ T cell infiltrates in early tumors possibly results from decreased RAGE-mediated MDSC and TAM immune suppression and inflammation. The increase in CD8+ cytotoxic T cells suggests improved immune response to tumors, potentially mediated by decreased MDSC and TAM immune suppression and inflammation.

### RFT01—Tumor Growth Inhibition

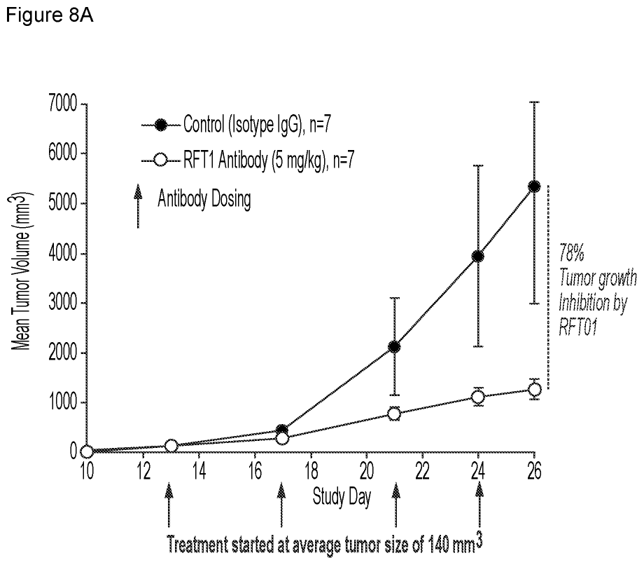

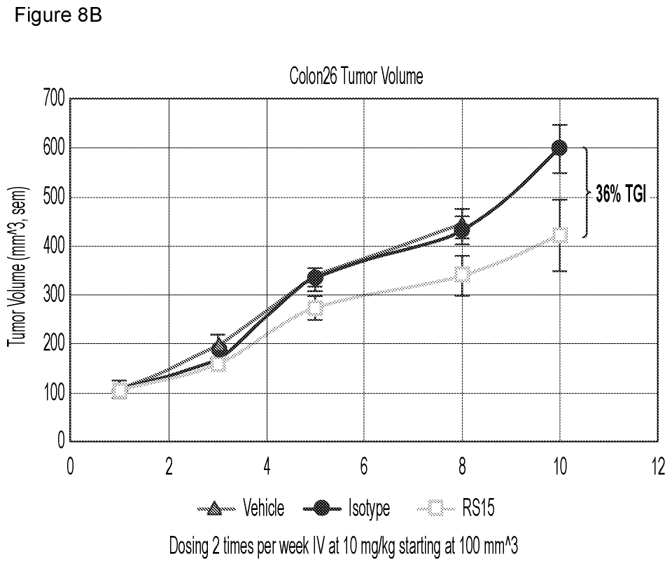

RFT01 treatment of CT26 tumor-bearing mice resulted in 78% tumor growth inhibition (FIG. 8A). FIG. 8B shows that dosing of RFT01 twice per week IV at 10 mg/kg starting with Colon-26 tumors of 100 mm³ resulted in a decrease in tumor volume over time.

TABLE 2

| SEQ | | |
|---|---|---|
| ID | | |
| NO: | Description | Sequence |
| 1 | Human RAGE | MAAGTAVGAWVLVLSLWGAVVGAQNITARIGEPLVLKCKGAPKK |
| | | PPQRLEWKLNTGRTEAWKVLSPQGGGPWDSVARVLPNGSLFL |
| | | PAVGIQDEGIFRCQAMNRNGKETKSNYRVRVYQIPGKPEIVDSA |
| | | SELTAGVPNKVGTCVSEGSYPAGTLSWHLDGKPLVPNEKGVSV |
| | | KEQTRRHPETGLFTLQSELMVTPARGGDPRPTFSCSFSPGLPR |
| | | HRALRTAPIQPRVWEPVPLEEVQLVVEPEGGAVAPGGTVTLTCE |
| | | VPAQPSPQIHWMKDGVPLPLPPSPVLILPEIGPQDQGTYSCVAT |
| | | HSSHGPQESRAVSISIIEPGEEGPTAGSVGGSGLGTLALALGILG |
| | | GLGTAALLIGVILWQRRQRRGEERKAPENQEEEEERAELNQSEE |
| | | PEAGESSTGGP |
| 2 | VH chain of RS15 | MEWSGVFIFLLSVTAGVHSQVQLQQSGAELVRPGTSLKVSCKAS |
| | | GYAFTNYLIEWVNQRPGQGLEWIGVINPGSGGTHYNEKFKGKAT |
| | | LTADKSSSTAYMQLSSLTSDDSAIYFCARSRAVRAMDYWGQGT |
| | | SVTV |
| 3 | VL chain of RS15 | MESQTQVFVYMLLWLSGVDGDIVMTQSQKFMSTSVGDRVSVTC |
| | | KASQNVGTNVAWYQQKPGQSPKSLIYSASYRYSGVPDRFTGSG |
| | | SGTDFTLTISNVQSEDLAEYFCQQYNSYFFTFGAGTKLELKR |
| 4 | CDR-H1 of RS15 | GYAFTNYL |
| 5 | CDR-H2 of RS15 | INPGSGGT |
| 6 | CDR-H3 of RS15 | ARSRAVRAMDY |
| 7 | CDR-L1 of RS15 | QNVGTN |
| 8 | CDR-L2 of RS15 | SAS |
| 9 | CDR-L3 of RS15 | QQYNSYFFT |
| 10 | VH chain of RS7 | MGWSSIIFFLVATATGVHSQVQLQQSGAELVRPGASVKISCKVS |
| | | DYTFTDYVMHCVKQSHGKSLEWIGVISTYYGYSDYNQKFKGKAT |
| | | MTVDKSSSTAYMDLGRLTSEDSAVYYCARGGVTTATGAMDYW |
| | | GQGTSVTVSSA |
| 11 | VL chain of RS7 | MKSHTQVFISILLWLYGADGNIVMTQSPKSMSMSVGERVTLTCK |
| | | ASENVVTYVSWYQQKPEQSPKLLIYGASNRYTGVPDRFTGSGS |
| | | ATDFTLTISSVQAEDLADYHCGQGYSYPYTFGGGTKLEIKRADAA |
| | | PTVSIFPPSS |
| 12 | CDR-H1 of RS7 | DYTFTDYV |
| 13 | CDR-H2 of RS7 | ISTYYGYS |
| 14 | CDR-H3 of RS7 | ARGGVTTATGAMDY |
| 15 | CDR-L1 of RS7 | ENVVTY |
| 16 | CDR-L2 of RS7 | GAS |
| 17 | CDR-L3 of RS7 | GQGYSYPYT |
| 18 | V domain of | LVLKCKGAPKKPPQRLEWKLNTGRTEAWKVLSPQGGGPWDSV |
| | human RAGE | ARVLPNGSLFLPAVGIQDEGIFRCQAMNRNGKETKS |
| 19 | C1 domain of | PEIVDSASELTAGVPNKVGTCVSEGSYPAGTLSWHLDGKPLVPN |
| | human RAGE | EKGVSVKEQTRRHPETGLFTLQSELMVTPARGGDPRPTFSCSF |
| | | SPGLPRHRA |
| 20 | C2 domain of | PGGTVTLTCEVPAQPSPQIHWMKDGVPLPLPPSPVLILPEIGPQD |
| | human RAGE | QGTYSCVATHSS |
| 21 | Signal sequence | MAAGTAVGAWVLVLSLWGAVVGA |
| | of human RAGE | |
| 22 | Cytoplasmic tail | RRQRRGEERKAPENQEEEEERAELNQSE |
| | of human RAGE | |
| 23 | Mouse RAGE | MPAGTAARAWVLVLALWGAVAGGQNITARIGEPLVLSCKGAPKK |
| | | PPQQLEWKLNTGRTEAWKVLSPQGGPWDSVARILPNGSLLLPA |
| | | TGIVDEGTFRCRATNRRGKEVKSNYRVRVYQIPGKPEIVDPASEL |
| | | TASVPNKVGTCVSEGSYPAGTLSWHLDGKLLIPDGKETLVKEET |
| | | RRHPETGLFTLRSELTVIPTQGGTHPTFSCSFSLGLPRRRPLNTA |
| | | PIQLRVREPGPPEGIQLLVEPEGGIVAPGGTVTLTCAISAQPPPQ |
| | | VHWIKDGAPLPLAPSPVLLLPEVGHEDEGTYSCVATHPSHGPQE |
| | | SPPVSIRVTETGDEGPAEGSVGESGLGTLALALGILGGLGVVALL |
| | | VGAILWRKRQPRREERKAPESQEDEEERAELNQSEEAEMPENG |
| | | AGGP |

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. An antibody specific for the V and/or C1 domains of the receptor for advanced glycation end products (RAGE), wherein the antibody comprises complementarity determining regions (CDR) comprising:
a) the amino acid sequences of SEQ ID NOs: 4, 5, and 6;
b) the amino acid sequences of SEQ ID NOs: 7, 8, and 9;
c) the amino acid sequences of SEQ ID NOs: 12, 13, and 14; or
d) the amino acid sequences of SEQ ID NOs: 15, 16, and 17.

2. The antibody according to claim 1, wherein a variable heavy (VH) region of the antibody has an amino acid sequence of SEQ ID NO. 2 or 10.

3. The antibody according to claim 1, wherein a variable light (VL) region of the antibody has an amino acid sequence of SEQ ID NO. 3 or 11.

4.-8. (canceled)

9. The antibody according to claim 1, wherein the antibody comprises CDRs of SEQ ID NOs: 4, 5, and 6.

10. The antibody according to claim 1, wherein the antibody comprises CDRs of SEQ ID NOs: 7, 8, and 9.

11. The antibody according to claim 1, wherein the antibody comprises CDRs of SEQ ID NOs: 12, 13, and 14.

12. The antibody according to claim 1, wherein the antibody comprises CDRs of SEQ ID NOs: 15, 16, and 17.

13. The antibody according to claim 1, wherein the antibody comprises the VH of SEQ ID NO: 2 and the VL of SEQ ID NO:3.

14. The antibody according to claim 1, wherein the antibody comprises the VH of SEQ ID NO: 10 and the VL of SEQ ID NO:11.

15. (canceled)

16. A method of inhibiting RAGE activity in a subject in need thereof, comprising administering to the subject an effective amount of an antibody according to any of claims 1-14.

17. The method according to claim 16, wherein inhibiting RAGE activity comprises treating cancer in the subject.

18. The method according to claim 16, wherein treating cancer comprises altering a tumor microenvironment, inhibiting vascularization of a tumor, inhibiting growth of a tumor, inhibition or decreased expression of growth factors and/or inflammatory factors within the tumor, decrease monocytic myeloid derived suppressor cells (MDSC), decrease inflammatory monocytes (iMonos), decrease M2 tumor associated macrophage (M2 TAM), increase M1 macrophage and M1 macrophage signaling, increase M1/M2 macrophage tumor infiltrates and/or signaling, M2 to M1 polarization, and/or to promote T cell activity and infiltration into the tumor.

19. The method according to claim 16, wherein inhibiting RAGE activity comprises reducing inflammation in the subject.

20. (canceled)

21. (canceled)

22. The method according to claim 16, wherein inhibiting RAGE activity comprises preventing or treating liver damage and/or fibrosis.

23. The method according to claim 16, wherein inhibiting RAGE activity comprises preventing or treating neovascularization in the eye.

24. The method according to claim 16, wherein inhibiting RAGE activity comprises preventing or treating respiratory diseases.

25. The method according to claim 16, wherein inhibiting RAGE activity comprises preventing or treating infections.

26. The method according to claim 16, further comprising administering an effective amount of a second medicament.

27.-29. (canceled)

30. The method according to claim 17, wherein the cancer is colorectal cancer.

31. A method of detecting a RAGE protein in a biological sample comprising contacting the sample with an anti-RAGE antibody according to claim 1, and detecting formation of a complex between the antibody and a RAGE protein in the sample.
